(19) United States
(12) Patent Application Publication
(10) Pub. No.: US 2024/0050471 A1
(43) Pub. Date: Feb. 15, 2024

(54) METHODS FOR ACTIVATING IMMUNE CELLS AND TREATING OCULAR NEOVASCULARIZATION

(71) Applicants: Rajendra Apte, St. Louis, MO (US); Joseph Lin, St. Louis, MO (US); Philip Ruzycki, St. Louis, MO (US)

(72) Inventors: Rajendra Apte, St. Louis, MO (US); Joseph Lin, St. Louis, MO (US); Philip Ruzycki, St. Louis, MO (US)

(73) Assignee: Washington University, St. Louis, MO (US)

(21) Appl. No.: 18/448,821

(22) Filed: Aug. 11, 2023

Related U.S. Application Data
(60) Provisional application No. 63/397,666, filed on Aug. 12, 2022.

(51) Int. Cl. A61K 35/15; A61P 27/02; A61K 45/06; A61K 38/17

(52) U.S. Cl. CPC A61K 35/15; A61P 27/02; A61K 45/06; A61K 38/1709

(57) ABSTRACT

The present disclosure provides for methods of activating an immune cell in a subject by administering to the subject a CYR61 activating agent, as well as methods of treating ocular neovascularization in a subject by administering a CYR61 activating agent to an immune cell to form a treated immune cell and administering the treated immune cell to the subject.

### CROSS-REFERENCE TO RELATED APPLICATIONS

This application claims priority from U.S. Provisional Application Ser. No. 63/397,666 filed on 12 Aug. 2022, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### Field of the Invention

The present disclosure generally relates to methods for the activation of immune cells and treatment of ocular neovascularization.

### SUMMARY OF THE INVENTION

Among the various aspects of the present disclosure is the provision of methods to activate immune cells and treat ocular neovascularization.

In one aspect, the present disclosure provides for a method of treating ocular neovascularization in a subject, the method comprising administering a CYR61 activating agent to an immune cell to form a treated immune cell; and administering the treated immune cell to the subject. In some embodiments, the CYR61 activating agent is CYR61. In some embodiments, the subject has macular degeneration, age-related macular degeneration (AMD), a retinal disease, or diabetic retinopathy. In some embodiments, the immune cell is selected from a macrophage and a monocyte. In some embodiments, the macrophage is selected from a monocyte-derived macrophage, a bone marrow-derived macrophage (BMDM), and a choroidal macrophage. In some embodiments, the subject has at least one choroidal neovascular (CNV) lesion. In some embodiments, administering the treated immune cell to the subject reduces CNV lesion size. In some 30 embodiments, the method further comprises administering to the subject an anti-VEGF treatment. In some embodiments, the CYR61 activating agent is administered to the immune cell for a duration of at least about 24 hours. In some embodiments, the treated immune cell is administered to the subject intravitreally. In some embodiments, administering the CYR61 activating agent to the immune cell upregulates expression of one or more genes selected from the group consisting of Saa3, Mmp9, Gm14493, Cfb, Mmp14, Slpi, Gm49383, Marco, Rps12I1, Pilra, Hp, CcI5, Nxpe5, Acod1, C3, Socs3, Mid1-ps1, Il1b, and combinations thereof.

Another aspect of the present disclosure provides for a method of activating an immune cell in a subject, the method comprising administering to the subject a CYR61 activating agent. In some embodiments, the subject has ocular neovascularization, macular degeneration, age-related macular degeneration (AMD), a retinal disease, or diabetic retinopathy. In some embodiments, the CYR61 activating agent is CYR61. In some embodiments, the immune cell is selected from a macrophage, a monocyte, a monocyte-derived macrophage, a bone marrow-derived macrophage (BMDM), and a choroidal macrophage. In some embodiments, the subject has at least one choroidal neovascular (CNV) lesion. In some embodiments, administering the CYR61 activating agent to the subject reduces CNV lesion size. In some embodiments, the method further comprises administering to the subject at least one of an anti-VEGF treatment and a photodynamic therapy. In some embodiments, the CYR61 activating agent is administered to the subject intravitreally. In some embodiments, administering the CYR61 activating agent to the subject upregulates immune cell expression of one or more genes selected from the group consisting of Saa3, Mmp9, Gm14493, Cfb, Mmp14, Slpi, Gm49383, Marco, Rps12I1, Pilra, Hp, CcI5, Nxpe5, Acod1, C3, Socs3, Mid1-ps1, Il1b, and combinations thereof. In some embodiments, administering the CYR61 activating agent to the subject upregulates endothelial cell expression in the subject of one or more genes selected from the group consisting of CCL2, CXCL6, CXCL1, TFRC, IL32, and combinations thereof. In some embodiments, administering the CYR61 activating agent to the subject downregulates endothelial cell expression in the subject of one or more genes selected from the group consisting of LYVE1, DEPP1, FCF1, AC020916.1, LINC02603, and combinations thereof.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 shows a schematic of experimental workflow in accordance with the present disclosure.

FIG. 2A-FIG. 2B is an exemplary embodiment showing cell heterogeneity of circulating immune cells and choroid in accordance with the present disclosure. FIG. 2A is a UMAP plot showing cell type heterogeneity of PBMCs. FIG. 2B is a dot plot showing marker gene expression for cell types in PBMCs.

FIG. 3A-FIG. 3C is an exemplary embodiment showing PBMC cell type distribution by patient and quality control metrics for PBMC scRNAseq data in accordance with the present disclosure. FIG. 3A includes stacked bar plots showing cell type distributions of PBMCs for each patient. FIG. 3B is a violin plot showing percentage of mitochondrial reads in each PBMC cell cluster. FIG. 3C is a violin plot showing the number of genes detected per cell in each PBMC cell cluster.

FIG. 4A-FIG. 4B is an exemplary embodiment showing PBMC cell types predicted by Azimuth in accordance with the present disclosure. FIG. 4A-FIG. 4B include UMAP plots showing PBMC cell type annotations predicted by Azimuth. Azimuth was used to automatically predict cell types using the pbmcrer reference. L1 and L2 cell type predictions are shown in FIG. 4A and FIG. 4B, respectively.

FIG. 5A-FIG. 5B is an exemplary embodiment showing cell heterogeneity of circulating immune cells and choroid in accordance with the present disclosure. FIG. 5A is a UMAP plot showing cell types that are shared and unique between PBMCs and choroid. FIG. 5B is a UMAP plot showing cell type heterogeneity of choroid and PBMCs, respectively.

FIG. 6A-FIG. 6E is an exemplary embodiment showing lineage-specific transcriptional signatures of immune cells localized to the choroid compared to those in the systemic circulation in accordance with the present disclosure. FIG. 6A is a stacked bar plot showing the number of up- and down-regulated genes for each immune cell lineage. Differentially expressed genes are those with pseudobulked |log₂ FC|>0.5 and adjusted p-value <0.05 (Wilcoxon rank sum test). FIG. 6B-FIG. 6E shows pathway analysis of genes activated in immune cells local to the choroid vs. those in peripheral circulation. The top 5 pathways for each immune cell lineage are shown, colored according to the horizontal axis labels in FIG. 6A.

FIG. 7A-FIG. 7B is an exemplary embodiment showing choroid-peripheral immune cell signaling dysregulated in age-related macular degeneration in accordance with the present disclosure. FIG. 7A-FIG. 7B include stacked bar plots showing the number of up- and down-regulated genes for each cell type in PBMCs and choroid, respectively. Differentially expressed genes are those with pseudobulked |log₂ FC|>0.5 and p-value <0.05 (Wilcoxon rank sum test).

FIG. 8A-FIG. 8B is an exemplary embodiment showing filtering of full choroid-PBMC signaling network in accordance with the present disclosure. FIG. 8A is a Venn diagram showing the number of paracrine ligand-receptor interactions that passed through each of 3 filtering criteria. FIG. 8B is an alluvial diagram showing the 242 interactions between choroidal cell types and peripheral immune cells that are dysregulated in AMD. The width of each connection that connects two cell types indicates the number of signaling interactions between the connected cell types.

FIG. 9 is an exemplary embodiment showing choroid-peripheral immune cell signaling dysregulated in age-related macular degeneration in accordance with the present disclosure. FIG. 9 shows a full intercellular network of ligand-receptor interactions that are dysregulated in AMD. The lines connecting icons are shaded by the LRscore calculated by SingleCellSignalR. Genes expressed by PBMC cell types are shown in color according to the legend whereas those expressed choroidal cell types are colored in gray and labeled in parentheses. V-shaped icons indicate receptor genes, whereas diamond icons indicate ligand genes.

FIG. 10 is an exemplary embodiment showing choroidal ligands or receptors dysregulated in AMD that can signal to circulating immune cells in accordance with the present disclosure. FIG. 10 includes violin plots showing 9 ligands/receptors that are dysregulated in choroidal cell types (pseudobulked |log₂ FC|>0.5 & p-values <0.05, Wilcoxon rank sum test). The specific cell types in which the genes are dysregulated are labeled in parentheses. Each point represents the average expression for one patient.

FIG. 11A-FIG. 11B is an exemplary embodiment showing THBS1 inhibits mouse CNV. FIG. 11A includes images showing laser-induced CNV lesions in wild type or Thbs1−/− mice. Images are en face view of the lesion on a flat-mounted choroid. Mice were injected with FITC-dextran to identify neovascular lesions. The white dashed line circumscribes the CNV lesion. FIG. 11B is a scatter dot plot showing CNV sizes in wild type or Thbs1^{-/-} mice (Mann Whitney test). Line indicates mean±SEM for 16-17 eyes from n=8-9 mice across 2 independent experiments. ***, p-value <0.001.

FIG. 12 is an exemplary embodiment showing choroidal ligands or receptors dysregulated in AMD that can signal to circulating immune cells in accordance with the present disclosure. FIG. 12 is a representative image of CYR61 localization in whole-mounted mouse CNV lesions in the laser injury-induced mouse model of wet AMD. Each image is an en face maximal projection of a confocal z-stack that spans the entire neovascular lesion across 19 slices. CD31 and CCR2 are markers of endothelial cells and monocyte-derived macrophages, respectively.

FIG. 13A-FIG. 13B is an exemplary embodiment showing CYR61 is strongly expressed in mouse choroidal neovascular lesions in accordance with the present disclosure. FIG. 13A includes representative images of CYR61 localization in transverse sections of control mouse retina and mouse CNV lesions. Each image is a maximal projection of a confocal z-stack that spans the entire tissue section across 14-16 slices. CD31 is a marker of endothelial cells. The white dashed line circumscribes the CNV lesion. Yellow arrowheads indicate areas of CYR61 and CD31 colocalization. FIG. 13B includes representative images of CYR61 localization in whole-mounted mouse CNV lesions in the laser injury-induced mouse model of wet AMD. Each image is an en face maximal projection of a confocal z-stack that spans the entire neovascular lesion across 21 slices. CD31 and CD45 are markers of endothelial cells and nucleated hematopoietic immune cells, respectively.

FIG. 14 is an exemplary embodiment showing Cyr61 transcript is localized to mouse CNV lesions in accordance with the present disclosure. FIG. 14 includes representative images of Cyr61 transcript localization in sections of mouse choroidal neovascular lesions. Each image is a maximal projection of a confocal z-stack that spans the entire tissue section across 10-15 slices. Cd31 is a marker of endothelial cells. Polr2a is a positive control. Dashed white line circumscribes the neovascular lesion. Yellow arrowheads indicate areas of Cyr61 and Cd31 colocalization.

FIG. 15A-FIG. 151 is an exemplary embodiment showing effect of CYR61 on endothelial cells and macrophages in accordance with the present disclosure. FIG. 15A is a heat map showing HUVEC genes whose expression is dysregulated after 24 hr incubation with CYR61 (FDR<0.05). FIG. 15B is a heat map showing THP1 gene expression changes after treatment with CYR61 (24 hr). Genes shown are those with FDR<0.05 and |log₂(fold-change)|>1.5 in THP1 cells treated with 5 μg/ml. FIG. 15C-FIG. 15D include pathway analysis of the 1,110 genes with FDR<0.05 upregulated in BMDMs following CYR61 treatment (24 hr, 0.5 μg/ml). The top 10 pathways, ranked by p-value are shown. FIG. 15E is a heat map showing genes dysregulated in BMDMs by CYR61 treatment (24 hr, 0.5 μg/ml) with FDR<0.05 and |log₂(fold-change)|>1.5. FIG. 15F includes bar graphs showing changes in the expression of 3 pro-inflammatory and 3 pro-angiogenic genes in BMDMs following 24 hr treatment with CYR61 (0.5 μg/ml). Statistical significance was assessed using Mann Whitney test. Bars show mean±SEM for n=4 wells/group (technical replicates). FIG. 15G is a Venn diagram showing the number of ligand-receptor interactions with a ligand dysregulated when BMDMs are treated with CYR61 and/or a receptor that is dysregulated in HUVECs when co-incubated with CYR61-activated BMDMs. These ligand-receptor interactions were from the LRdb database. FIG. 15H shows fifty-eight macrophage-to-endothelial cell ligand-receptor interactions that are dysregulated when macrophages are activated by CYR61. FIG. 151 shows macrophage ligands dysregulated by CYR61 that can signal to endothelial cells. *, p value <0.05.

FIG. 16A-FIG. 16B is an exemplary embodiment showing CYR61-activated macrophage genes are upregulated in choroidal macrophages compared to circulating monocytes in accordance with the present disclosure. FIG. 16A includes violin plots showing top CYR61-responsive genes that were dysregulated in choroidal macrophages compared to circulating monocytes (pseudobulked p-value <0.05, Wilcoxon rank sum test). Each point represents average macrophage or monocyte gene expression in one patient. FIG. 16B is a violin plot showing the CYR61-responsive gene CCL5 is upregulated in choroidal macrophages in AMD compared to choroidal macrophages in control patients (pseudobulked p-value <0.05, Wilcoxon rank sum test). Each point represents average macrophage CCL5 expression in one patient.

FIG. 17A-FIG. 17D is an exemplary embodiment showing CYR61 inhibits choroidal neovascularization in accordance with the present disclosure. FIG. 17A includes images showing immunostaining for CYR61 and CD31 in mouse CNV lesions in eyes injected intravitreally with negative control or anti-Cyr61 locked nucleic acid (2 μl/eye, 150 PM). Each image is an en face maximal projection of a confocal z-stack that spans the entire neovascular lesion across 12-13 slices. FIG. 17B is a bar graph showing quantification of mean CYR61 fluorescence intensity. Bars for each group indicate mean±SEM for images of n=7-8 CNV lesions from 5-6 eyes from n=3 mice. Statistical significance was assessed using a Mann Whitney test. FIG. 17C includes images showing laser-induced CNV lesions in mice administered negative control or anti-Cyr61 locked nucleic acid via intravitreal injection (2 μl/eye, 150 μM). Images are en face view of the lesion on a flat-mounted choroid. Mice were injected with FITC-dextran to visualize neovascular lesions. The white dashed line circumscribes the CNV lesion. FIG. 17D is a scatter dot plot showing relative 15 CNV sizes in mice receiving intravitreal injection of negative control or anti-Cyr61 locked nucleic acid. Sizes of CNV lesions were averaged by eye and then normalized to the average negative control CNV size in each experiment. Line indicates mean±SEM for 31 eyes from 3 independent experiments. n≥15 mice across 3 independent experiments. Statistical significance was assessed using a Mann Whitney test. *, p-value <0.05.

FIG. 18 is a schematic showing a proposed model of CYR61 inhibition of choroidal neovascularization in accordance with the present disclosure.

FIG. 19 contains images showing age-related macular degeneration (AMD) in accordance with the present disclosure.

FIG. 20 is a plot showing CYR61 ligand does not affect CNV size in accordance with the present disclosure.

FIG. 21 is a plot showing secreted factors from CYR61-treated bone marrow derived macrophages does not affect CNV size in accordance with the present disclosure.

FIG. 22 is a plot showing CYR61-treated macrophages injected intravitreally inhibit CNV lesion size in accordance with the present disclosure.

### DETAILED DESCRIPTION OF THE INVENTION

The present disclosure is based, at least in part, on the discovery that CYR61 contributes to the unique activation state of macrophages localized to the choroidal microenvironment and CYR61-treated bone marrow-derived macrophages (BMDMs) inhibited choroidal neovascular (CNV) lesions. As shown herein, methods are described to activate immune cells and treat ocular neovascularization.

Age-related macular degeneration (AMD) is the leading cause of blindness in people >50 years old in developed nations. About 1.7 million people in the US have AMD. The wet form of AMD (which involves CNV) affects about 200,000 people per year in the US. CNV lesions are a major clinical manifestation of age-related macular degeneration (AMD) as well as diabetic retinopathy. The two most common treatments for neovascular or wet AMD are anti-VEGF injections and photodynamic therapy. Both treatments can slow down the progression of the disease but not cure it.

CYR61 is a secreted protein and linked to tumorigenesis as well as angiogenesis, however, its role in AMD is not well-understood. Previous studies have suggested CYR61 as being proangiogenic and that depletion of CYR61 (by knockdown) reduces VEGF expression and inhibits CNV formation (see e.g., Sun et al. (2017) Sci Rep. 7, 14925). Surprisingly, as disclosed herein, CYR61 demonstrates antiangiogenic effects and in exemplary embodiments is useful in treatment of retinal diseases like AMD and diabetic retinopathy.

Treatment of Ocular Neovascularization and Associated Diseases

Provided is a process of treating ocular neovascularization in a subject in need thereof, wherein the subject is administered a CYR61 activating agent or a CYR61 activated agent-treated immune cell. In exemplary embodiments the CYR61 activating agent is CYR61 or at least comprises CYR61.

Ocular neovascularization refers to abnormal growth of blood vessels in the eye. Ocular neovascularization is characteristic of retinal and choroidal vascular diseases, which are common causes of vision loss in developed countries. These diseases include retinal vascular diseases, characterized by leakage or neovascularization from retinal vessels, and subretinal neovascularization, characterized by new vessel growth into the outer retina and subretinal space. Examples of retinal vascular disease include retinopathy, retinal vein occlusions, and retinopathy of prematurity. Examples of subretinal neovascularization disease include retinal angiomatous proliferation (RAP), choroidal neovascularization, defects or deposits in Bruch's membrane, age-related macular degeneration (AMD), ocular histoplasmosis, and pathologic myopia.

Methods described herein are generally performed on a subject in need thereof. A subject in need of the therapeutic methods described herein can be a subject having, diagnosed with, suspected of having, or at risk for developing ocular neovascularization. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a CYR61 activating agent or a CYR61 activating agent-treated immune cell is, for example, an amount that would cause the desired therapeutic effect in a subject while minimizing undesired side effects. In various embodiments, an effective amount of a CYR61 activating agent or a CYR61 activating agent-treated immune cell described herein can substantially inhibit CNV lesions, slow the progress of CNV lesions, or limit the development or size of CNV lesions.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic or intravitreal, buccal, or rectal administration.

When used in the treatments described herein, a therapeutically effective amount of a CYR61 activating agent or a CYR61 activating agent-treated immune cell can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat ocular neovascularization or CNV lesions.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes reversing, or delaying the appearance of clinical symptoms in a mammal afflicted with the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a CYR61 activating agent or a CYR61 activating agent-treated immune cell can occur as a single event or over a time course of treatment. For example, a CYR61 activating agent or a CYR61 activating agent-treated immune cell can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for CNV lesions or ocular neovascularization.

A CYR61 activating agent or a CYR61 activating agent-treated immune cell can be administered simultaneously or sequentially with another agent, such as an anti-VEGF treatment, photodynamic therapy, antibiotic, an anti-inflammatory, or another agent. For example, a CYR61 activating agent or a CYR61 activating agent-treated immune cell can be administered simultaneously with another agent, such as an anti-VEGF treatment, photodynamic therapy, antibiotic, or an anti-inflammatory. Simultaneous administration can occur through the administration of separate compositions, each containing one or more of a CYR61 activating agent or a CYR61 activating agent-treated immune cell, an 25 anti-VEGF treatment, photodynamic therapy, antibiotic, an anti-inflammatory, or another agent. Simultaneous administration can occur through the administration of one composition containing two or more of a CYR61 activating agent or a CYR61 activating agent-treated immune cell, an anti-VEGF treatment, photodynamic therapy, antibiotic, an anti-inflammatory, or another agent. A CYR61 activating agent or a CYR61 activating 30 agent-treated immune cell can be administered sequentially with an anti-VEGF treatment, photodynamic therapy, antibiotic, an anti-inflammatory, or another agent. For example, a CYR61 activating agent or a CYR61 activating agent-treated immune cell can be administered before or after the administration of an anti-VEGF treatment, photodynamic therapy, antibiotic, an anti-inflammatory, or another agent.

In some embodiments, the methods of the present disclosure provide for administering an anti-VEGF treatment to a subject. Such treatments are known in the art and commonly used for the treatment of ocular neovascularization and related diseases. 5 For example, an anti-VEGF treatment can be brolucizumab (Beovu), aflibercept (Eylea), ranibizumab (Lucentis or SUSVIMO), faricimab-svoa (VABYSMO), or bevacizumab (Avastin).

In some embodiments, the methods of the present disclosure provide for administering a photodynamic therapy to a subject. Photodynamic therapy, also known as photobiomodulation therapy (PBT), is commonly used for treatment of ocular neovascularization and related diseases. For example, a photodynamic therapy may include administering alight-sensitive medicine such as verteporfin (Visudyne) and exposing the eye to laser-emitted light.

CYR61 Activating Agents

As described herein, CYR61 expression has been implicated in various diseases, disorders, and conditions associated with ocular vascularization. As such, activation of CYR61 can be used for the treatment of such conditions. For example, a CYR61 activation agent can activate immune cells towards a disease-modulating phenotype and upregulate genes involved in inflammation, angiogenesis, cell adhesion, or immune cell recruitment.

A CYR61 activation agent in accordance with the present disclosure can be any agent that increases expression or activity of CYR61. In exemplary embodiments, the CYR61 activation agent is CYR61 or comprises CYR61.

In some embodiments, a CYR61 activation agent is a gene expression construct or vector designed to express or overexpress CYR61. Design and production of such constructs or vectors is routine in the art.

In other embodiments, H2S, polysulfides, the slow-release H2S generator GYY4137, and the polysulfide donor Na2S activate the CYR61 promoter. HMPSNE (2-[(4-hydroxy-6-methylpyrimidin-2-yl)sulfanyl]-1-(naphthalen-1-yl)ethan-1-one (and AOAA (aminooxyacetic acid) increase CYR61 protein levels (see e.g., Ascencao et al. (2022) Redox Biol. Vol. 56).

In further embodiments, sphingosine 1-phosphate (S1P) stimulates the expression of CYR61 at both the mRNA and protein levels. As an additional embodiment, RhoA, MKK6, or MKK3 increase activity at the CYR61 promoter (see e.g., Han et al. (2003) Eur. J. Biochem. Vol. 270).

In yet other embodiments, overexpression of the S1P receptors S1P₁ or S1P₂ increase expression of CYR61 (Young and Van Brocklyn (2007) Exp Cell Res. Vol. 313).

In yet further embodiments, CYR61 is activated by agonists of G protein-coupled receptors (GPCRs) such as thrombin, prostaglandins E₂ and F_{2α}, and sphinogosine-1-phosphate. Factors that bind to the serum response element (SRE) in the CYR61 promoter, such as serum response factor (SRF) may also be used as CYR61 activation agents. Myocardin-related transcriptional activator (MRTF-A), p38 mitogen-activated protein kinase (MAPK), and transcription factor Egr-1 enhance CYR61 expression. As another example, hypoxic conditions have been shown to induce CYR61 expression (see e.g., Lau (2011) Cell Mol Life Sci. Vol. 68).

In yet additional embodiments, Ang II, the α/β-adrenergic agonist norepinephrine, the α₁-selective agonist phenylephrine, and TNF-α increase CYR61 expression (see e.g., Hilfiker-Kleiner et al. (2004) Circulation, Vol. 109).

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single-specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of computer simulation programs known in the art that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | lle, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing getting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic or intravitreal, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a treated immune cell, CYR61, CYR61 activating agent, media, and immune cells. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water and sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrates, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Targeting Cell Type-Specific, Choroid-Peripheral Immune Signaling to Treat Age-Related Macular Degeneration

This Example describes how CYR61 contributes to the unique activation state of macrophages localized to the choroidal microenvironment, activates macrophage-to-endothelial signaling pathways that can modify angiogenesis, and provides a protective signal that inhibits pathologic ocular angiogenesis.

Summary

Age-related macular degeneration (AMD) is a leading cause of blindness in industrialized nations featuring lipid-rich deposits underneath the retina and neovascularization of the ocular choroidal vasculature. Although systemic immunity plays a critical role in this pathogenic neovascularization, the molecular signals that recruit immune cells to the eye in AMD remain poorly defined. Herein, peripheral blood mononuclear cells from 65 individuals including AMD and controls were prospectively profiled using single cell RNA sequencing (scRNAseq), which were integrated with existing choroid scRNAseq data. A network of 242 interactions between choroidal cell types and circulating immune cells that are dysregulated in AMD was generated which included known AMD-relevant genes such as VEGF receptor 2. Of novel significance, CYR61 was identified as upregulated in choroidal veins and may signal to several immune cell types via integrins. In mice, it was validated that CYR61 was abundant in endothelial cells within choroidal neovascular lesions in close proximity to CCR2+ monocyte-derived macrophages. Mechanistically, CYR61 activated macrophage anti-angiogenic gene expression and macrophage-to-endothelial signaling pathways. Ocular knockdown of Cyr61 in mice increased neovascular lesion size, indicating CYR61 inhibits choroidal neovascularization. The present disclosure demonstrates the enormous potential of utilizing multi-tissue human data to identify disease-relevant and therapeutically modifiable targets.

Introduction

Age-related macular degeneration (AMD) is a multifactorial disease and the leading cause of blindness in the industrialized world. Early AMD is detected clinically by pathognomonic lipid-rich deposits underneath the neurosensory retina called drusen. Vision loss can occur later-either progressively from atrophic neurodegeneration (dry AMD) and/or rapidly from pathogenic angiogenesis (wet AMD). In dry AMD, atrophy of the retinal pigment epithelium, photoreceptors, and choriocapillaris leads to slowly progressive vision loss. On the other hand, wet AMD involves the formation of new blood vessels from the ocular choroidal vasculature that underlies and nourishes the neurosensory retina (i.e., choroidal neovascularization, CNV). Though less common, wet AMD accounts for 80-90% of sudden and severe vision loss from AMD due to hemorrhage. The advent of inhibitors against vascular endothelial growth factor (VEGF) has revolutionized the care for wet AMD but some patients remain under-/nonresponsive. Therefore, it is crucial to identify VEGF-independent pathways involved in the progression or control of AMD.

There is substantial evidence that the immune system plays a role in the pathogenesis of AMD. Genome-wide association studies have demonstrated that polymorphisms in immune-related genes such as complement factor H increase an individual's risk for AMD. Additionally, pathologic evidence indicates that immune cells, especially macrophages, are enriched in choroid and/or Bruch's membrane of patients with AMD. The recruitment of immune cells to CNV lesions has been recapitulated in animal models of AMD. Macrophage activation state and aging appears to factor into whether these recruited cells promote or inhibit the disease process. Though abundant evidence exists highlighting the critical role of immune cells in the pathogenesis of AMD, the molecular signaling pathways that recruit/activate immune cells in this disease remain poorly defined.

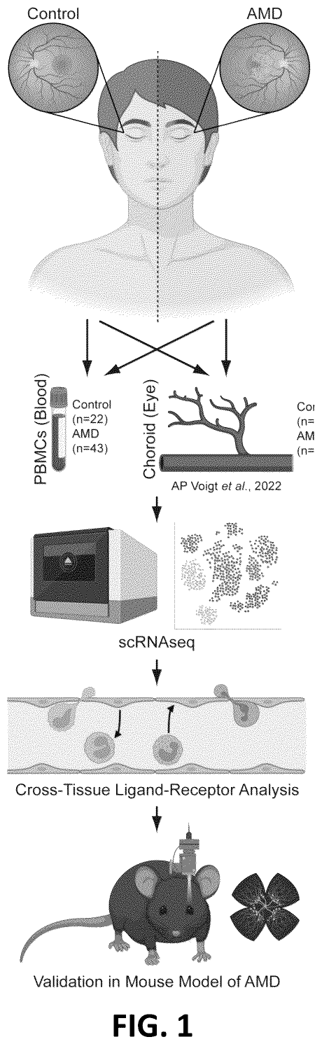

Previous examinations of peripheral immune cells have focused on expression of cytokines and chemokines and found dysregulated expression of several inflammatory molecules in AMD. As described herein, single cell RNA sequencing (scRNAseq) was utilized to agnostically elucidate the cell type-specific molecular signaling that recruits immune cells to the eye in AMD and/or regulates their role in AMD. Peripheral blood mononuclear cells (PBMCs) from 43 patients with AMD and 22 controls were profiled by scRNAseq (see e.g., FIG. 1). The PBMC dataset was harmonized with a recently published scRNAseq dataset of choroid cells from AMD patients (n=11) and controls (n=10). This integrated, multi-tissue dataset enabled identification of i) the lineage-specific transcriptional signatures of immune cells localized to the choroidal niche compared to those in the systemic circulation; and ii) cell type-specific transcriptional signatures in AMD for both peripheral immune cells and cells of the choroidal stroma. In addition, a network was generated of 242 cross-tissue signaling interactions perturbed in AMD between circulating peripheral immune cells and cells in the choroidal vasculature. These include KDR, APOE, LGALS1, and THBS1 which are known to be relevant to AMD or CNV. Of novel significance, CYR61 was identified as upregulated in choroidal veins in AMD and signaling to integrin receptors expressed by many peripheral immune cell types. In the laser injury-induced CNV mouse model that recapitulates CNV in human AMD, CYR61 is highly expressed in endothelial cells of CNV lesions. Mechanistically, CYR61 robustly activated macrophage gene expression including activation of pro-inflammatory/anti-angiogenic markers. Many macrophage genes found to be responsive to CYR61 were also upregulated in choroidal macrophages compared to peripheral circulating monocytes, suggesting that CYR61 could contribute, at least in part, to the unique activation state of macrophages localized to the choroidal microenvironment. In addition, herein is shown that CYR61 activates macrophage-to-endothelial signaling pathways that can modify angiogenesis. Finally, shown herein is that knockdown of ocular Cyr61 by intravitreal injection of a locked nucleic acid into mouse eyes significantly increased CNV size, suggesting that CYR61 provides a protective signal that inhibits pathologic ocular angiogenesis. The present disclosure unravels one of potentially many complex cell-to-cell interactions connecting systemic immunity, deregulation of lipid homeostasis, and the local choroidal vascular bed in AMD. These findings may inform the development of novel treatment strategies to complement existing anti-VEGF therapies by using multi-tissue transcriptomic data to identify unique cross-tissue interactions.

Results

Transcriptional Heterogeneity of Peripheral Immune Cells

To better understand the transcriptional cell heterogeneity of circulating immune cells in AMD, control and AMD patients were recruited (see TABLE 1 for demographic information).

TABLE 1
Demographic and clinical characteristics of control
and AMD patients included in this study.

| | | PBMC | | | Choroid | P |
|---|---|---|---|---|---|---|
| | Control | AMD | P value | Control | AMD | value |
| Age, median | 74.5 | 77 | <0.05^{a} | 76.5 | 90 | <0.01^{a} |
| (range) | (63-95) | (60-94) | | (61-83) | (77-92) | |
| Sex, N | | | | | | |
| Male | 9 | 18 | 0.94^{b} | 6 | 5 | 0.51^{b} |
| Female | 13 | 25 | | 4 | 6 | |
| AMD status, N | | | | | | |
| No AMD | 22 | 0 | | 10 | 0 | |
| Early AMD | 0 | 8 | | 0 | 9 | |
| Geographic | 0 | 6 | | 0 | 0 | |
| atrophy only | | | | | | |
| Choroidal | 0 | 16 | | 0 | 2 | |
| neovascularization | | | | | | |
| only | | | | | | |
| Geographic | 0 | 13 | | 0 | 0 | |
| atrophy and | | | | | | |
| choroidal | | | | | | |
| neovascularization | | | | | | |

^{a}2-tailed Mann-Whitney U test
^{b}X² test

Blood was collected by venous blood draw, and PBMCs were isolated and cryopreserved in liquid nitrogen. When sufficient samples were collected, PBMCs were thawed and up to 8 samples were prepared at a time for scRNAseq on the 1 Ox Genomics platform. After thawing, PBMC samples had a median viability of 69%.

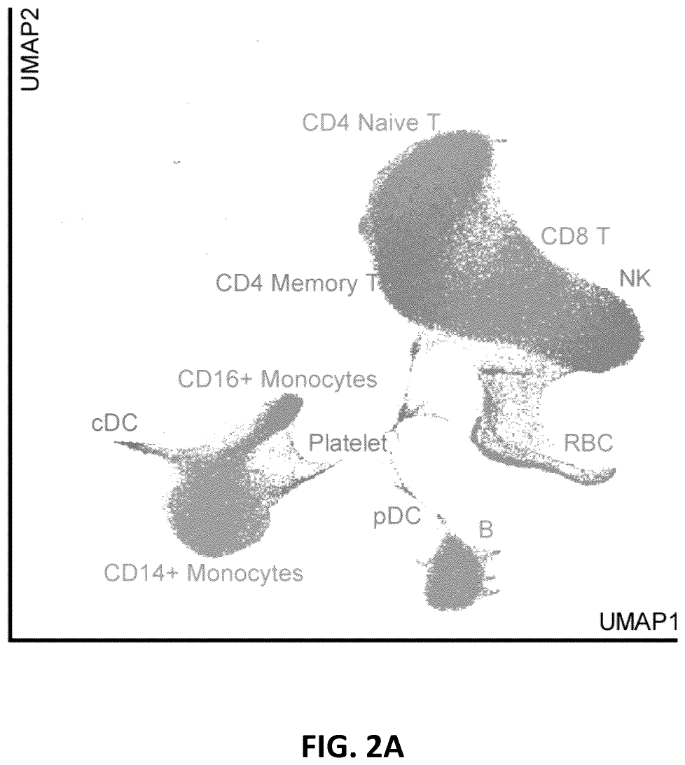

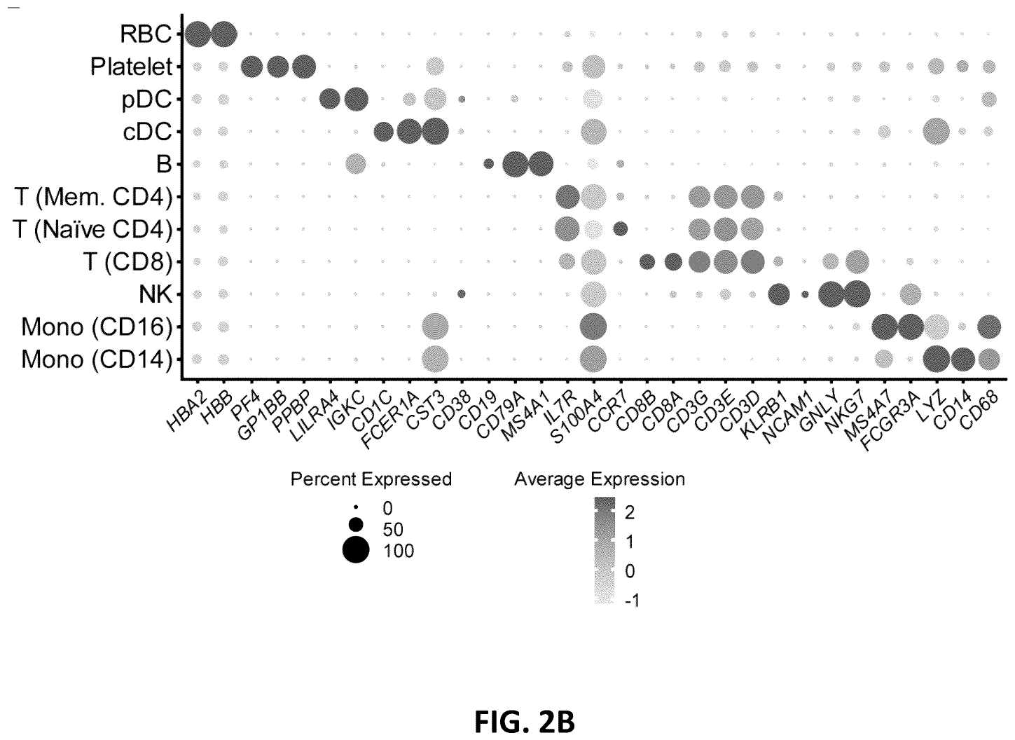

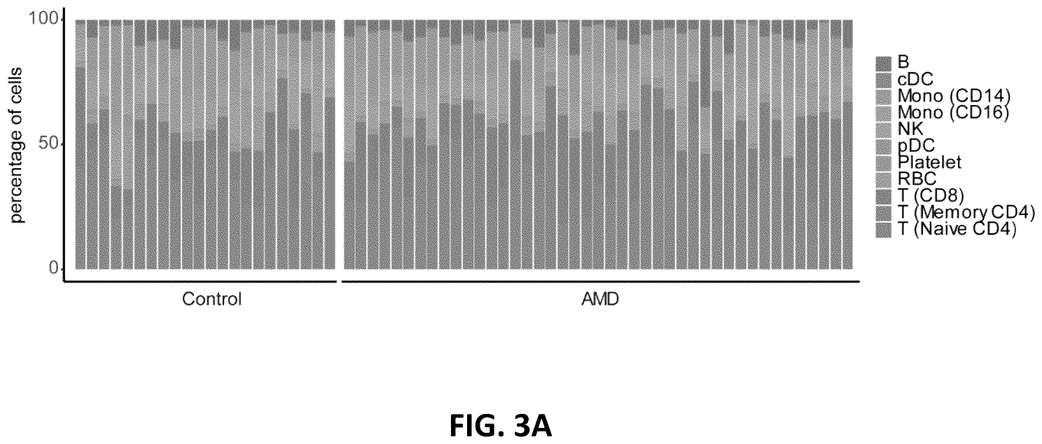

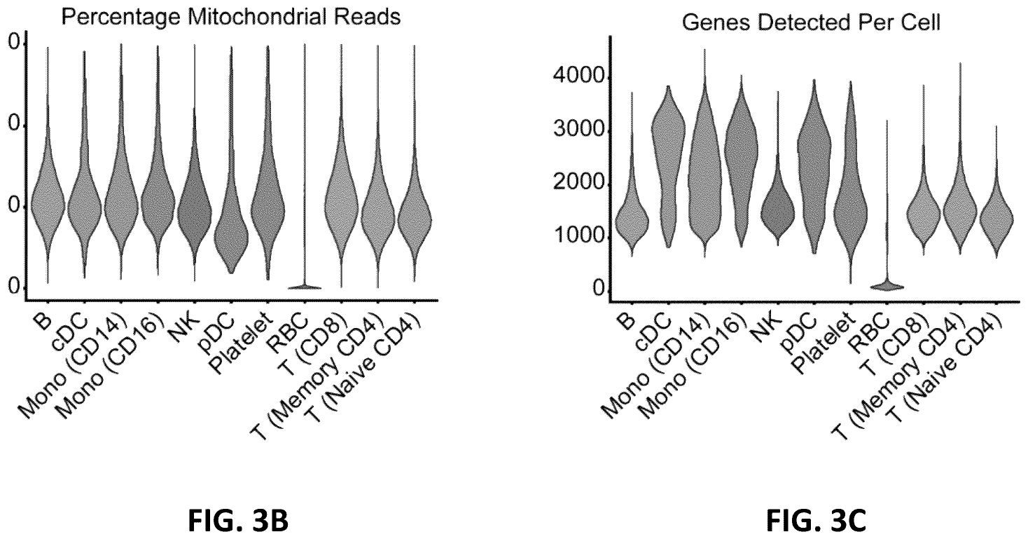

In total, transcriptomes were obtained for 161,315 PBMCs which were clustered into 11 major cell types (see e.g., FIG. 2A-FIG. 2B and FIG. 3A-FIG. 30). These included: classical and activated monocytes (CD14+ and CD16+, respectively); classical and plasmacytoid dendritic cells (cDCs and pDCs); platelets; red blood cells (RBCs); B cells; natural killer cells (NK); naïve and memory CD4+ T cells; and CD8+ T cells (see e.g., FIG. 2A-FIG. 2B). The distribution of these 11 cell types was largely similar between control and AMD patients, though there was some sample-to-sample variation (see e.g., TABLE 2 and FIG. 3A).

TABLE 2
Peripheral blood immune cell relative frequencies in
control and AMD patients.

| | Control (n = 22) | AMD (n = 43) |
|---|---|---|
| B | 4.8% (1.8-11.7) | 5.5% (0.7-35.1) |
| cDC | 0.4% (0.1-1.1) | 0.4% (0.0-1.4) |
| Monocytes (CD14) | 12.8% (3.7-39.5) | 13.8% (2.1-25.3) |
| Monocytes (CD16) | 3.8% (0.5-11.1) | 3.2% (0.3-7.9) |
| NK | 8.9% (2.1-24.0) | 9.2% (4.1-23.8) |
| pDC | 0.3% (0.1-1.0) | 0.4% (0.0-0.9) |
| Platelet | 0.7% (0.2-4.6) | 0.7% (0.1-3.9) |
| RBC | 3.1% (0.7-25.1) | 2.0% (0.0-17.2) |
| T (CD8) | 13.3% (6.0-40.2) | 19.9% (7.1-43.7) |
| T (Memory CD4) | 19.6% (10.3-28.0) | 20.1% (7.7-31.3) |
| T (naïve CD4) | 18.1% (8.2-35.8) | 19.6% (5.5-35.4) |
| Median (min-max) | | |

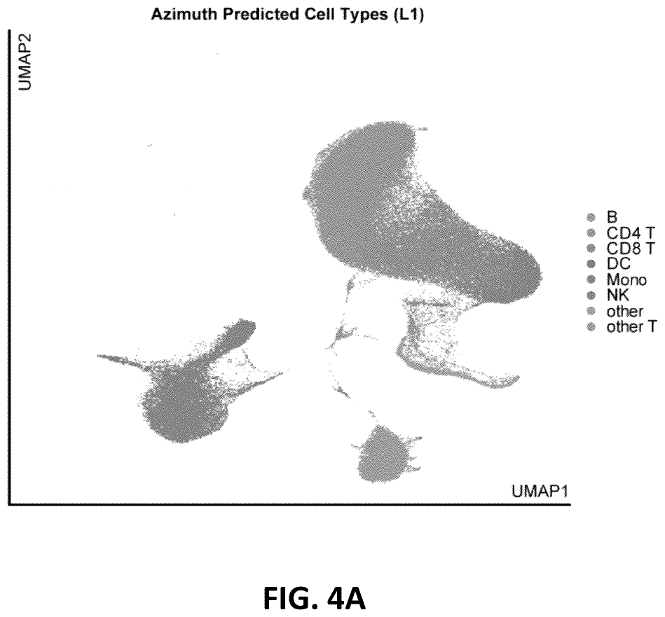

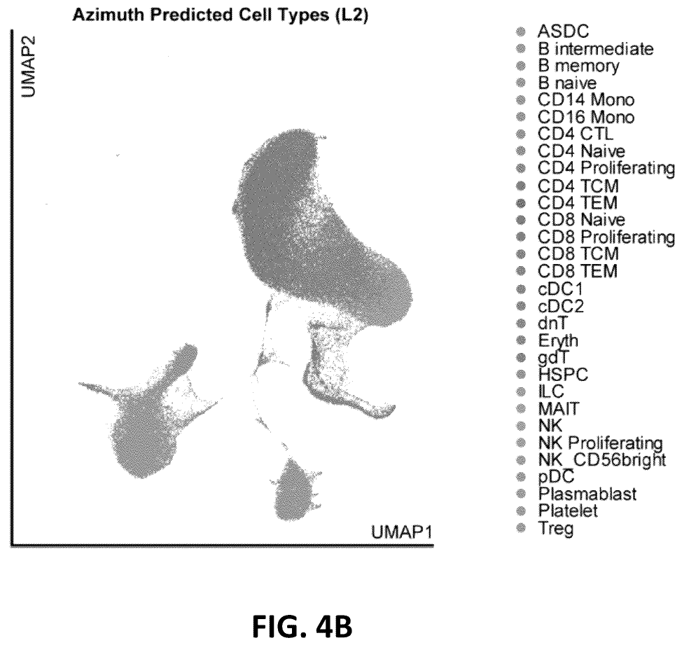

The manual cell type annotations were largely consistent to those predicted by Azimuth (see e.g., FIG. 4A-FIG. 4B).

Integration of PBMC and Choroid Datasets

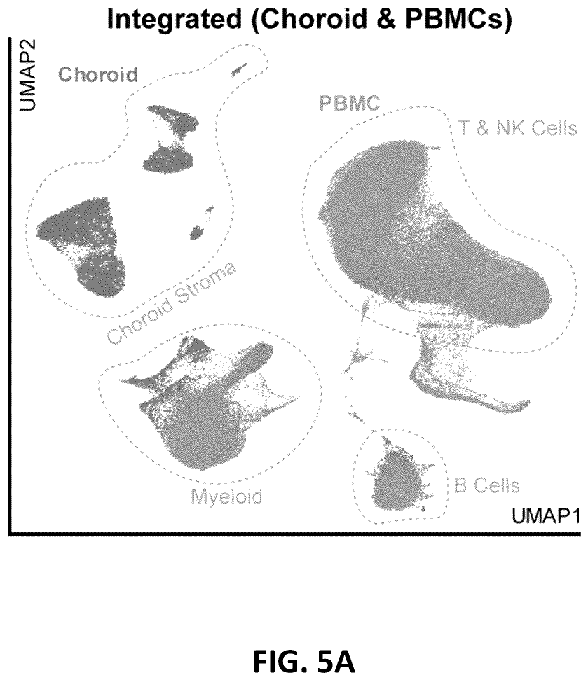

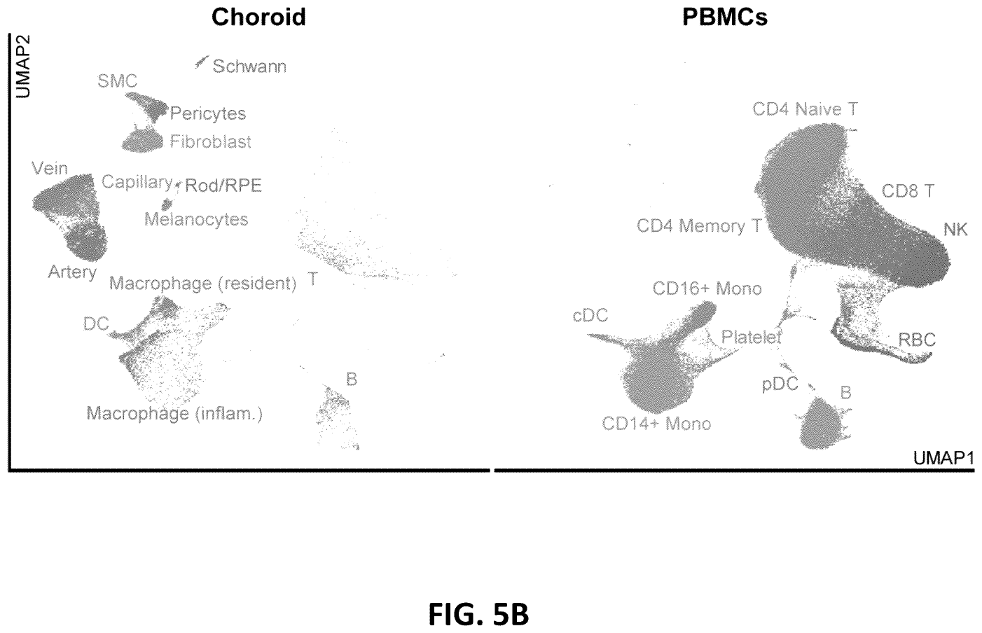

The PBMC dataset generated in-house was harmonized with an existing scRNAseq dataset of 30,416 human choroid cells (n=10 controls, n=11 AMD patients). This dataset was generated from dissociated from human foveal tissue containing choroid and retinal pigment epithelium (RPE) with positive selection for CD31 to enrich for endothelial cells. When the two datasets are visualized together, there were immune cell clusters that were shared between the two datasets and stromal cell clusters that were unique to the choroid dataset (see e.g., FIG. 5A). For the shared immune cell types, the annotated PBMC cell types were consistent with the choroid cell types annotated independently by Voigt et al. (see e.g., FIG. 5B and Voigt et al. (2022) Hum Mol Genet, ddac043). For instance, the choroidal T cells overlapped with peripheral blood T cells; choroidal B cells overlapped with peripheral blood B cells; and choroidal myeloid cells overlapped with peripheral blood myeloid cells (i.e., macrophages, dendritic cells, monocytes). In addition to these cell types that are common to both tissues, there were some cell types in the choroid dataset that were largely absent from the PBMC dataset, as expected (see e.g., FIG. 5A-FIG. 5B). These are: smooth muscle cells (SMCs), pericytes, fibroblasts, endothelial cells (venous, arterial, and choriocapillaris), melanocytes, Schwann cells, and rod/RPE cells (see e.g., FIG. 5A-FIG. 5B). Therefore, scRNAseq can distinguish cell types that are unique to one tissue or shared between two different tissues.

Lineage-Specific Transcriptional Hallmarks Distinguishing Systemic and Choroidal Immune Cells

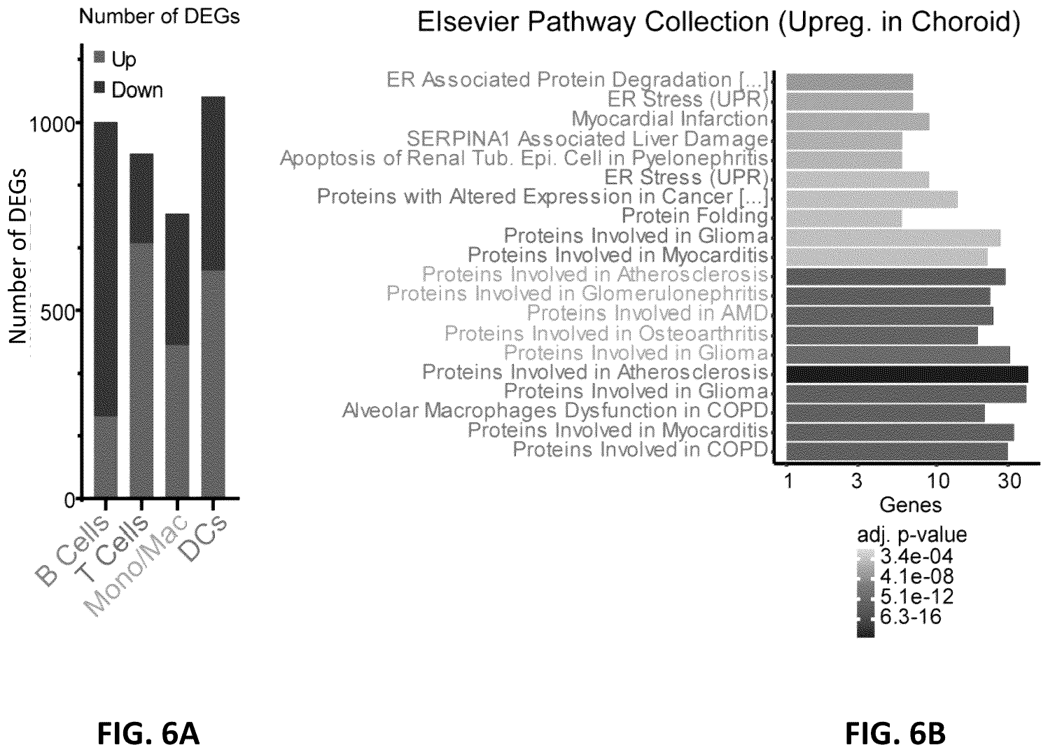

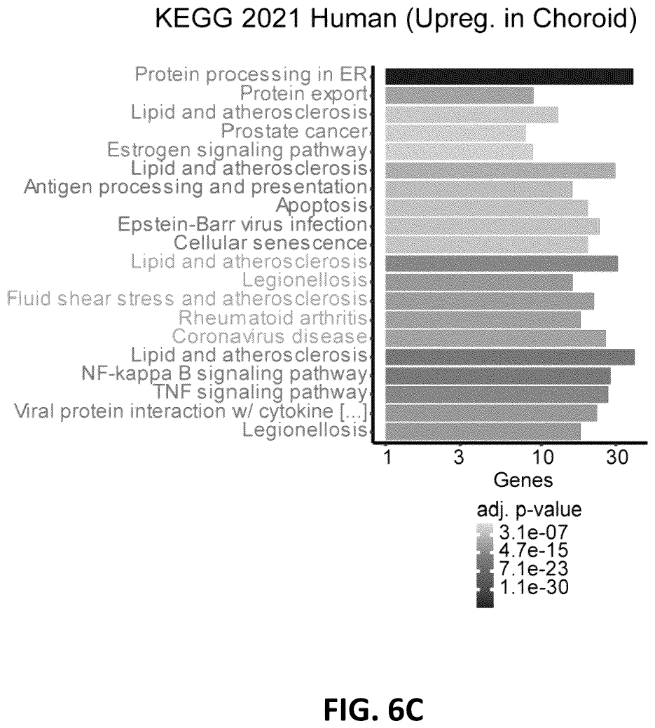

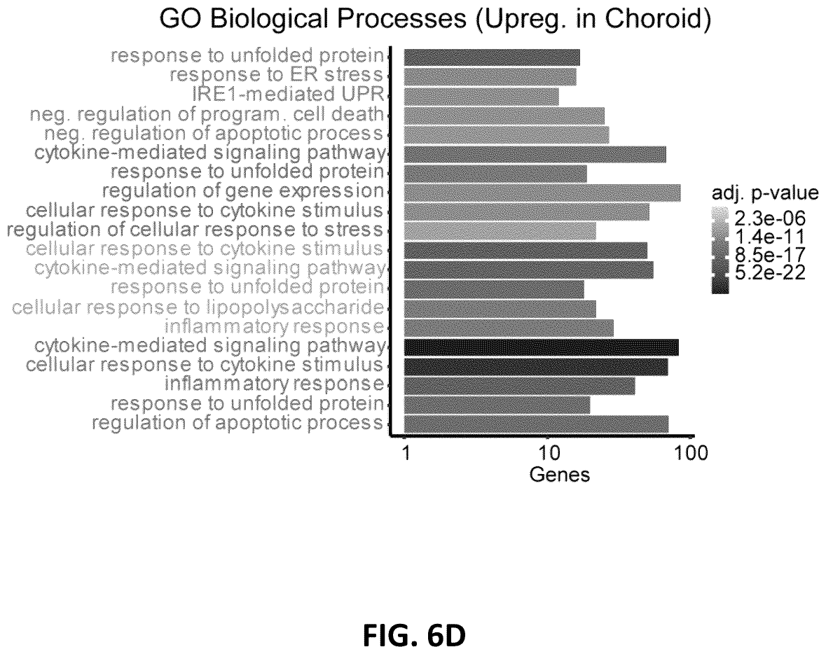

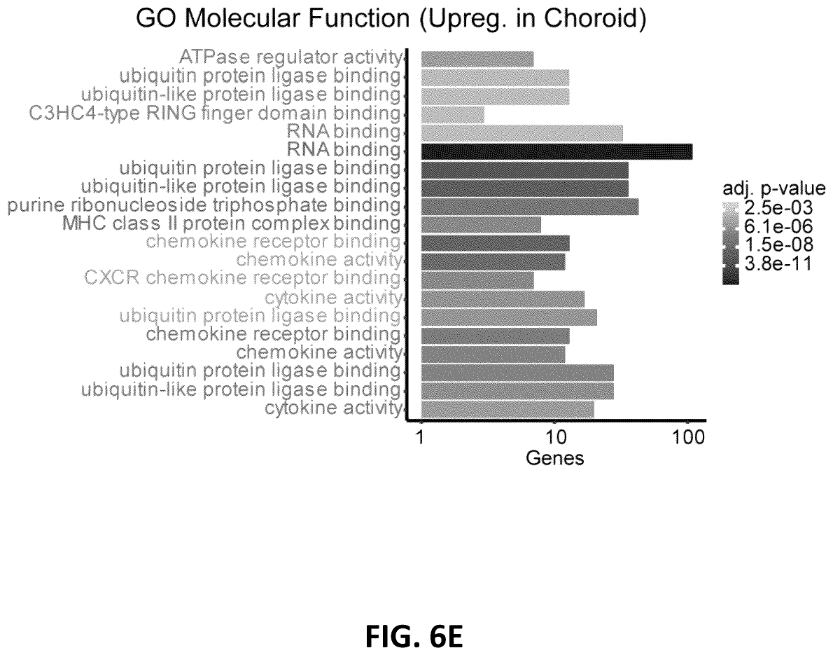

Since abundant evidence indicates that immune cells are recruited to the eye in neovascular AMD, gene expression signatures were first searched for that distinguished immune cell lineages localized to the choroid compared to peripheral cells of the same lineage in the systemic circulation. Comparisons were performed for the following cell lineages: monocyte/macrophages (macrophages in choroid vs. monocytes in circulation); B cells (in choroid vs. circulation); T cells (T cells in choroid vs. CD4+/CD8+ T cells in circulation); and dendritic cells (dendritic cells in choroid vs. cDCs in circulation) (see e.g., FIG. 6A). There were ˜1,000 differentially expressed genes (DEGs) with |log₂FC|>0.5 and adjusted p_values <0.05 identified for each immune cell lineage (see e.g., FIG. 6A). In order to determine whether DEGs upregulated in immune cells localized to the choroid were characteristic of specific pathways, pathway analysis was run on the following gene set libraries: Elsevier Pathway Collection; KEGG; GO Molecular Function; and GO Biological Process (see e.g., FIG. 6B-FIG. 6E). In general, genes activated in choroidal immune cell populations were related to inflammation; chemokine signaling; vessel diseases; lipid response; and cellular senescence (see e.g., FIG. 6B-FIG. 6E). Notably, the pathway “Proteins Involved in Age-Related Macular Degeneration” was one of the top pathways for monocytes/macrophages, suggesting a critical role for these local choroidal macrophages in AMD (see e.g., FIG. 6B). Therefore, the multi-tissue scRNAseq dataset highlights that immune cells localized to the choroidal microenvironment appear to be transcriptionally activated compared to those in the systemic circulation. These comparisons between immune cells localized to the ocular microenvironment to circulating immune cells support that there may be ocular signals that recruit and activate immune cells in the eye.

Cell Type-Specific Transcriptional Signatures in AMD

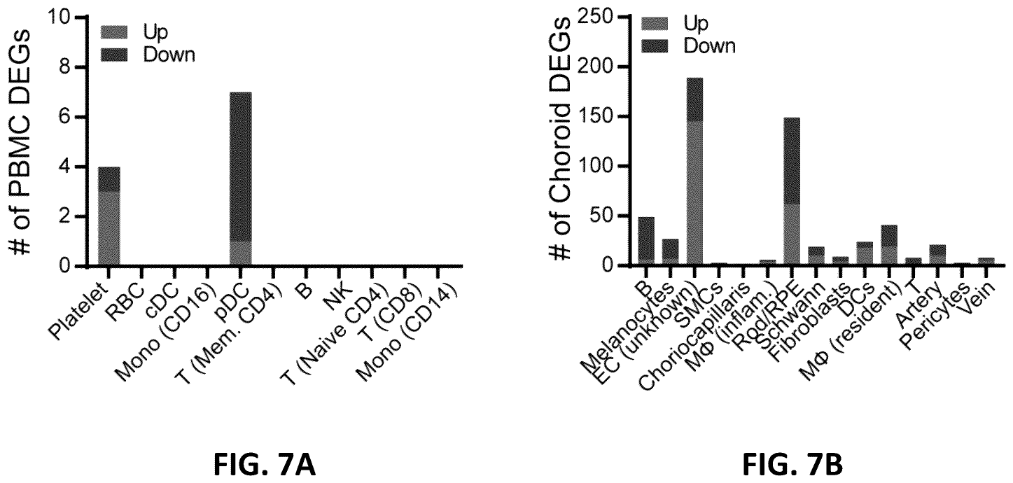

Gene expression-pseudobulked by patient was compared for each cell type in control vs. AMD patients for PBMCs and choroid (see e.g., FIG. 7A-FIG. 7B). In general, there were more gene expression changes in the choroid than in PBMCs, consistent with the fact that AMD is a disease whose manifestations are localized to the eye (see e.g., FIG. 7A-FIG. 7B). Among PBMC cell types, only pDCs and platelets had any statistically significant DEGs (unadjusted p-value <0.05, |log₂FC|>0.5). However, pDCs and platelets were a very rare cell type detected at 0.3-0.4% and 0.7%, respectively, which could contribute to noisy gene expression profiles in the comparisons. Therefore, the dataset indicates that at the transcriptional level, PBMCs are similar in AMD and control patients. This is in contrast to previous studies of AMD showing that there are perturbations to expression of several cytokines and chemokines in PBMCs. This discrepancy in findings could be methodological, as limited sequencing depth in scRNAseq could preclude detection of subtle changes. Additionally, many cytokines are regulated post-transcriptionally and changes at these later regulation steps could be overlooked in the scRNAseq dataset.

Ligand-Receptor Interactions Between Choroid and Circulating Immune System

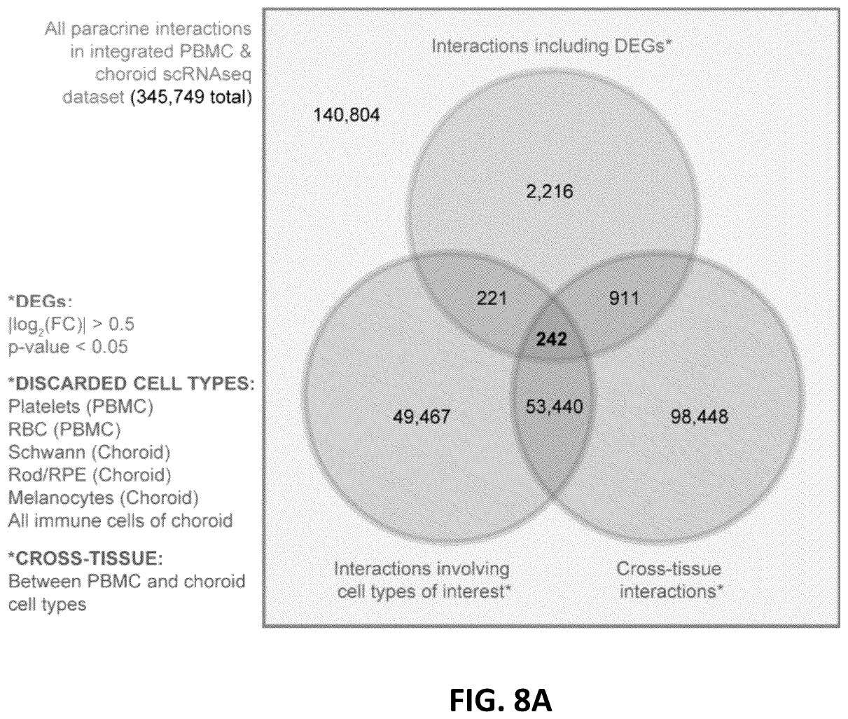

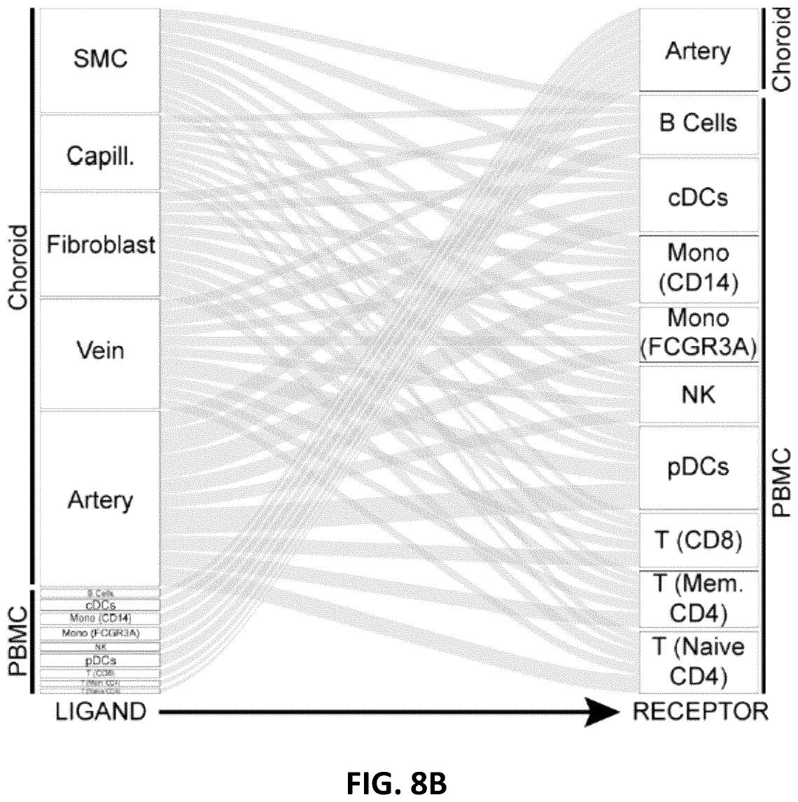

To identify potential signals that could recruit immune cells to the eye and activate them in AMD, ligand-receptor scoring was used to identify all paracrine interactions in the harmonized PBMC & choroid dataset with the tool SingleCellSignalR. In brief, this computes LR_{score} (ranging from 0-1) for known ligand-receptor pairs from the expression of the ligand in one cell population and the expression of its cognate receptor in another cell population. Ligand-receptor pairs that have high expression in two distinct cell populations will have high LR_{score}, suggesting that there may be paracrine signaling between these two cell populations. Among the 26 discrete cell populations in the dataset, there were 345,749 potential paracrine interactions identified with LR_{score}>0.5 (see e.g., FIG. 8A).

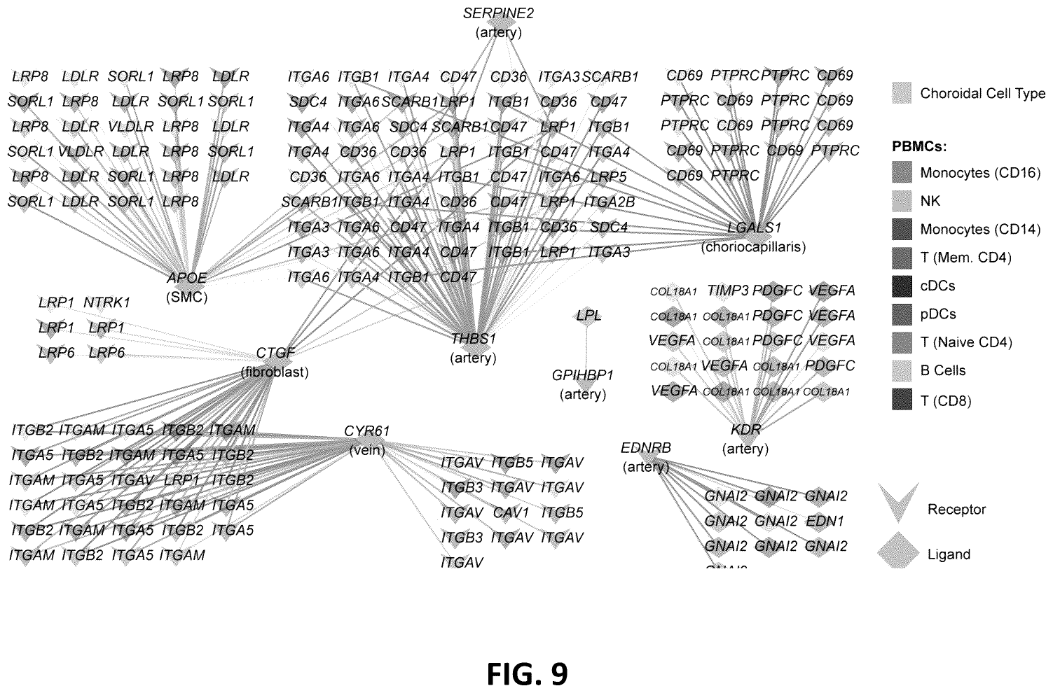

To narrow down this enormous list of interactions for ones that may be relevant to systemic immune cell recruitment to the choroid and their in situ activation in AMD, 3 filters were applied (see e.g., FIG. 8A). First, interactions involving cell types that were not of interest were discarded. Any interactions involving platelets, RBCs, Schwann cells, rod/RPE cells, melanocytes, and all immune cells in the choroid were excluded. Second, interactions were only included for which there was dysregulation of the ligand and/or receptor in the appropriate cell type in AMD. Finally, because interactions that could recruit systemic immune cells to the choroid in AMD were primarily of interest, interactions were filtered for only those that were cross-tissue (e.g., between choroid cell type and PBMC cell type). The intersection of these 3 filters revealed 242 interactions between circulating immune cells and the choroid that were perturbed in AMD patients (see e.g., FIG. 8A-FIG. 8B and FIG. 9).

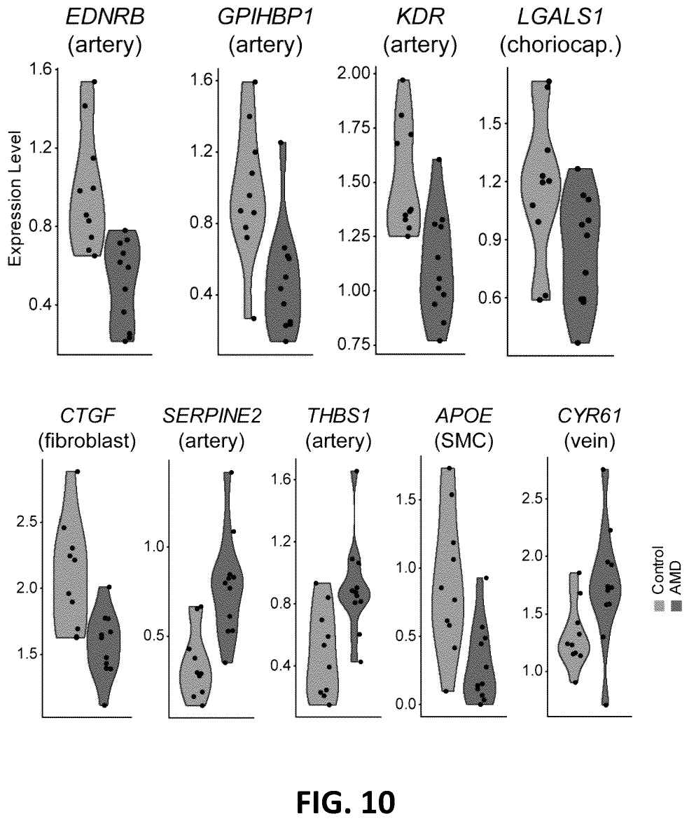

Unsurprisingly, all of these 242 interactions were dysregulated at the level of the choroid since gene expression in PBMCs was largely consistent between control and AMD patients, suggesting a role for the tissue micromilieu in regulating immune effector cell responses. The dysregulated genes relevant to PBMC-choroid interactions were: APOE (in SMCs), CTGF (in fibroblasts), THBS1 (in artery), LGALS1 (in choriocapillaris), CYR61 (in vein), GPIHBP1 (in artery), KDR (in artery), EDNRB (in artery), and SERPINE2 (in artery) (see e.g., FIG. 9 and FIG. 10). Notably, KDR is a receptor for VEGF, a pro-angiogenic factor that promotes CNV and is currently the only therapeutic target in the clinical management of neovascular AMD. The dataset indicates that KDR was downregulated in choroidal arterial endothelial cells (see e.g., FIG. 10). Of the remaining 8 genes, 3 are upregulated (CYR61, SERPINE2 and THBS1) and the rest are downregulated in AMD (see e.g., FIG. 10). Overall, these 8 genes are related to lipid homeostasis (APOE and GPIHBP1) or angiogenesis/inflammation (CTGF, THBS1, LGALS1, CYR61, EDNRB, and SERPINE2). In fact, THBS1 and LGALS1 have already been shown to inhibit and promote CNV, respectively, in previous studies. In general, these dysregulated PBMC-choroid interactions seem directly relevant to the cardinal clinical hallmarks of AMD: disrupted lipid homeostasis and inflammation/CNV.

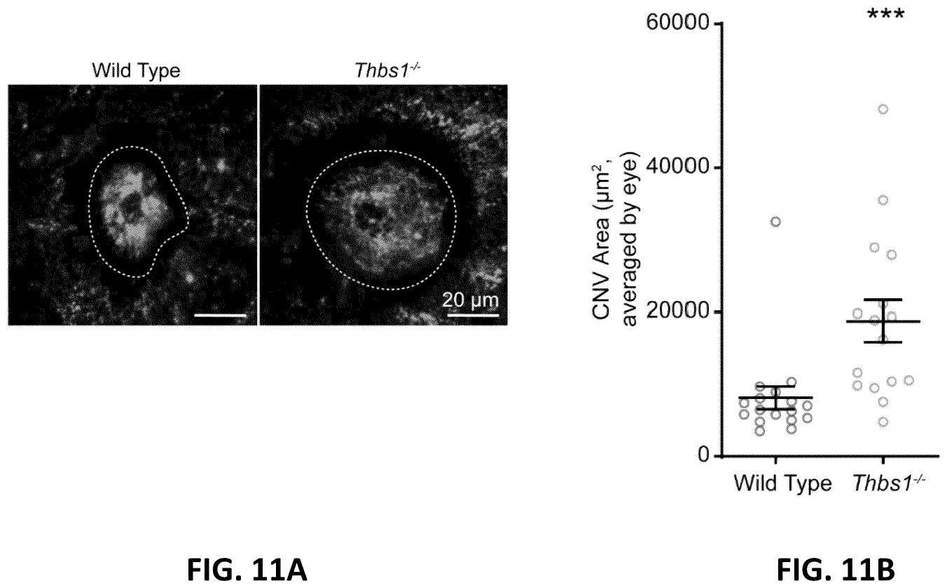

To provide independent validation that genes identified in this work are relevant to aspects of AMD pathogenesis, the role of THBS1 in CNV was tested by performing laser injury-induced CNV in mice deficient for this gene (see e.g., FIG. 11A-FIG. 11B). In this mouse model, a laser beam disrupts Bruch's membrane and initiates choroidal neovascular sprouting. Consistent with previous work, animals with genetic deletion of Thbs1 exhibited statistically significant ˜2.7 times larger CNV sizes (see e.g., FIG. 11A-FIG. 11B).

CYR61 is Strongly Expressed by Endothelial Cells in Murine CNV Lesions

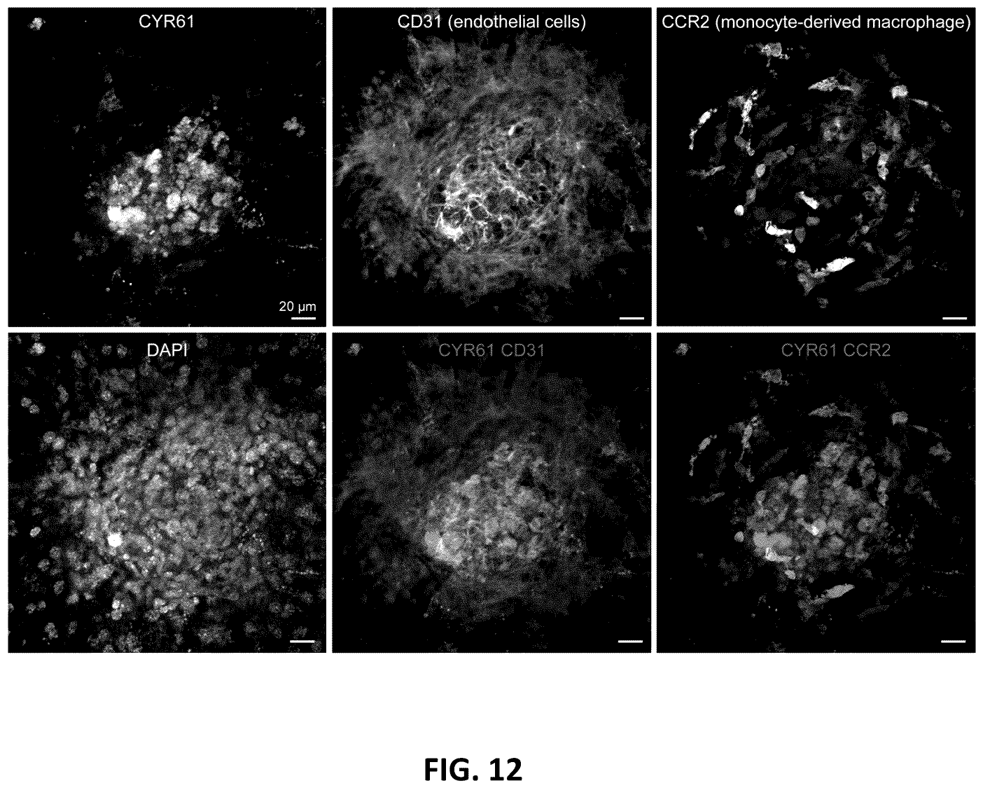

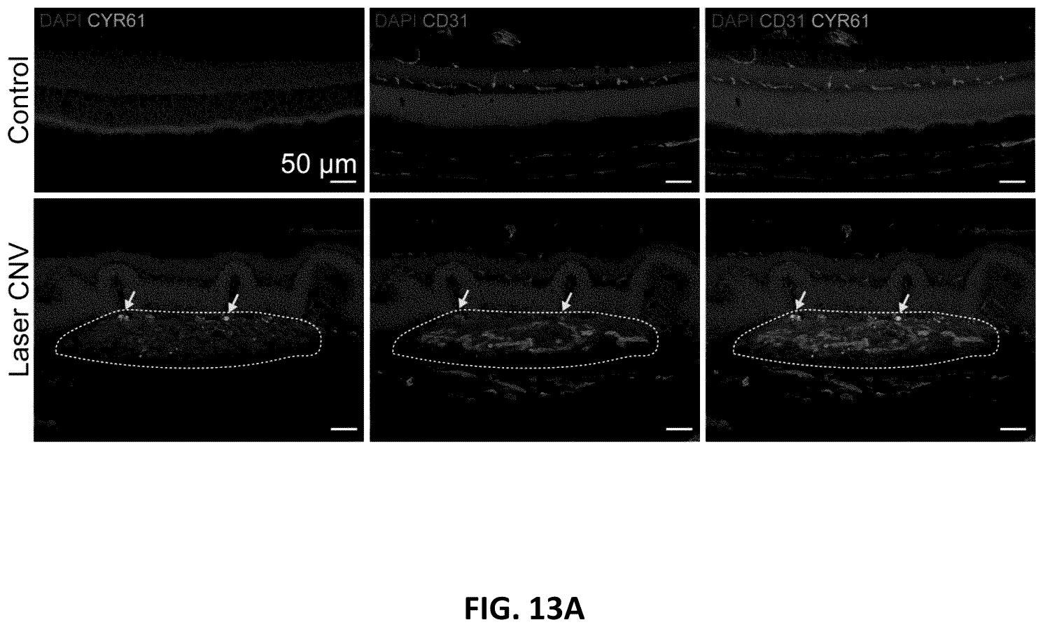

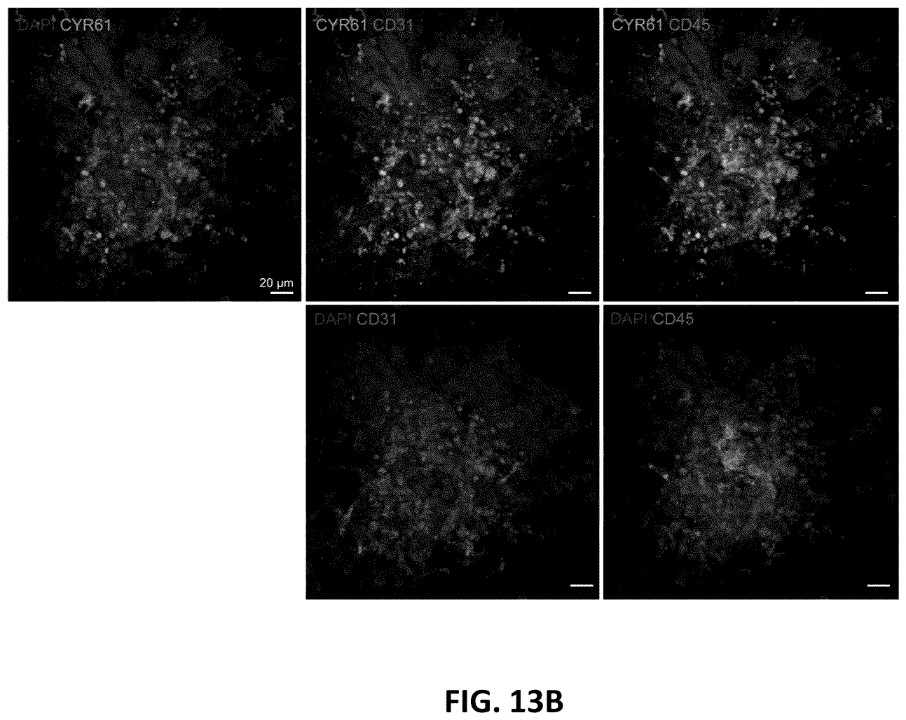

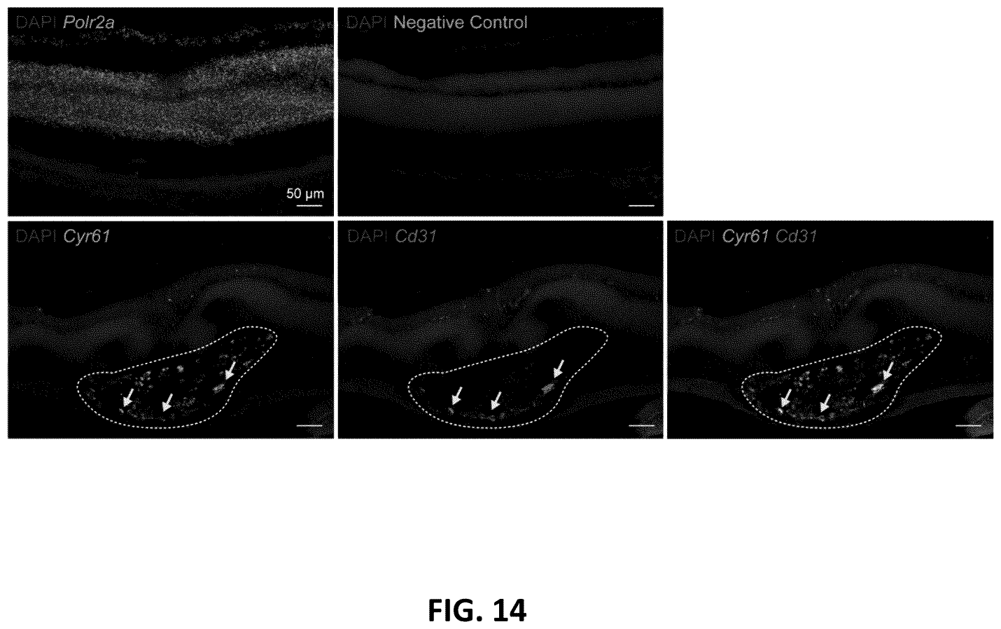

From the results of the ligand-receptor analysis, CYR61, whose role in CNV is less well established, was chosen for further investigation. CYR61 is a secreted matricellular protein that binds to integrin receptors and regulates angiogenesis in many contexts. It is expressed during cardiovascular development and is also expressed during inflammation and tissue repair in many tissues in adulthood. CYR61 can bind to αVβ/αVβ5/αMβ2 on macrophages, promoting adhesion and cytokine production; and can also bind to αVβ/αVβ5 on endothelial cells, promoting adhesion and growth factor-induced proliferation. Due to CYR61's ability to bind multiple relevant cell types and exert cell type-specific effects, it was determined to play a role in CNV, a complex process involving competing pro- and anti-angiogenic efforts. To first determine whether upregulation of CYR61 is associated with choroidal neovascularization, it was evaluated whether CYR61 was localized to CNV lesions in the laser injury-induced mouse model of CNV. CYR61 was strongly expressed in CD31+ endothelial cells in CNV lesions but absent in control mouse eyes (see e.g., FIG. 12, FIG. 13A-FIG. 13B, and FIG. 14). The ligand-receptor scoring indicated that CYR61 expressed by choroidal venous endothelial cells could signal to several immune cell populations, including monocytes which can differentiate into tissue macrophages (see e.g., FIG. 9). Consistent with previous studies, there were CD45+ hematopoietic immune cells in the CNV lesion and these immune cells were in close proximity to CYR61 protein (see e.g., FIG. 13B). More specifically, CCR2+ monocyte-derived macrophages were present in the CYR61-rich neovascular lesion (see e.g., FIG. 13B), consistent with previous studies. Taken together, these findings support that CYR61 expressed specifically by endothelial cells in choroidal neovascular lesions could signal to nearby immune cells, including monocyte-derived macrophages.

CYR61 Mildly Activates Endothelial Cell Gene Expression Relevant to Recruitment and Activation of Immune Cells

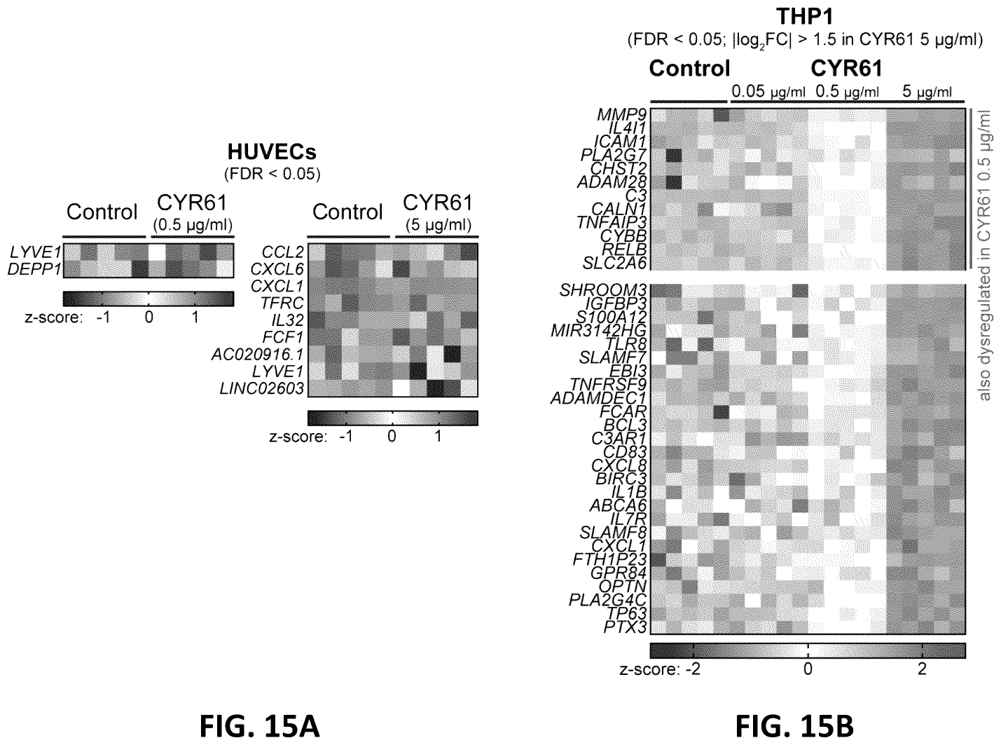

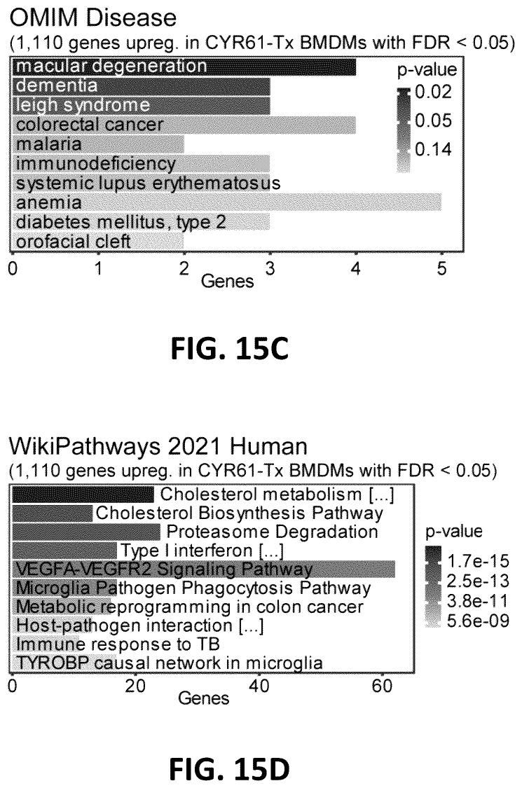

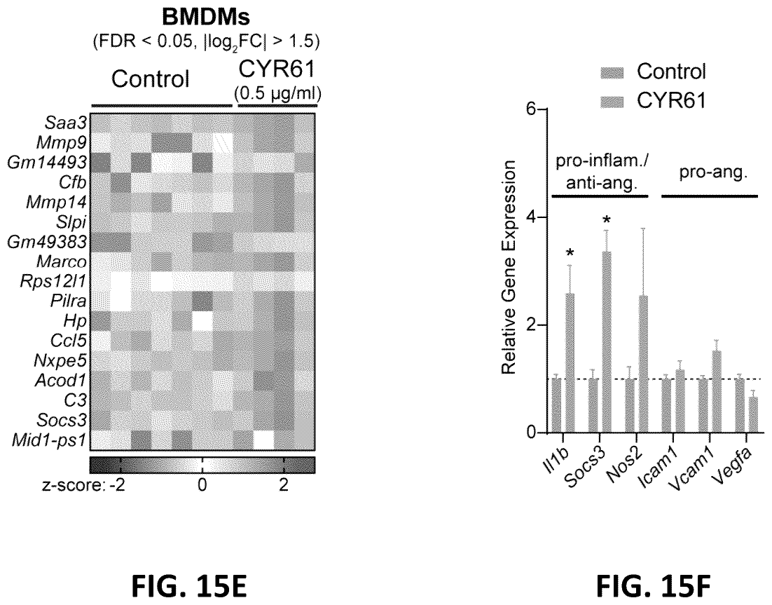

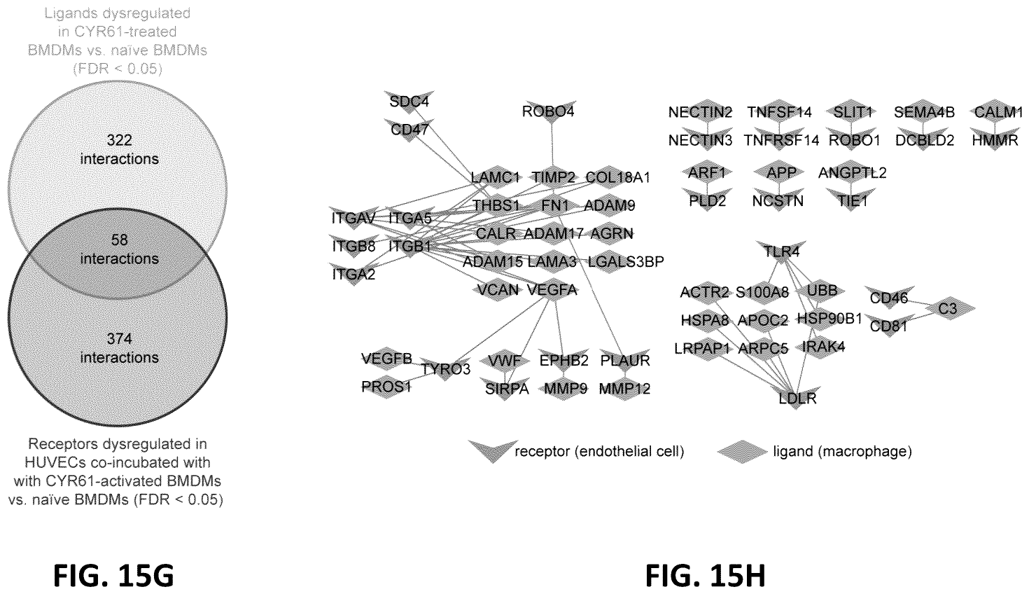

Since CYR61 is known to exert multiple effects on endothelial cells, it is possible that it could have autocrine effects on endothelial cells. To agnostically identify CYR61 effects on endothelial cells, RNA sequencing was performed to broadly identify transcriptional changes when human umbilical vein endothelial cells (HUVECs) are treated with exogenous CYR61 protein. Control HUVECs were compared to HUVECs treated with 0.05, 0.5, or 5.00 μg/ml CYR61 for 24 hr (n=5/group). An average of 67.1 million paired-end reads were obtained per sample and of these, an average of 66.8 million reads (99.6%) mapped to the human genome. DEG analyses were performed to compare control vs. 0.05 μg/ml CYR61; control vs. 0.5 μg/ml CYR61; and control vs. 5.0 μg/ml CYR61. In general, there were very mild transcriptional changes with <10 genes in each of these comparisons with FDR<0.05. A dose-dependent effect was noted on the number of genes with FDR<0.05 (0 DEGs for 0.05 μg/ml, 2 DEGs for 0.5 μg/ml, 9 DEGs for 5.0 μg/ml). The two genes dysregulated by moderate 0.5 μg/ml dose of CYR61 were both downregulated: LYVE1 (↓1.4×) and DEPP1 (↓1.4×) (see e.g., FIG. 15A). The nine genes dysregulated by the higher 5.0 μg/ml dose of CYR61 included 5 upregulated genes (CCL2 ↑1.8×; CXCL6 ↑1.7×; CXCL1 ↑1.5×; TFRC ↑1.5×; and IL32 ↑1.2×) and 4 downregulated genes (FCF1 ↓1.3×; AC020916.1 ↓1.3×; LYVE1 ↓1.3×; and LINC02603 ↓14×) (see e.g., FIG. 15A). LYVE1 was the only gene that was dysregulated by both CYR61 concentrations, but did not exhibit dose-dependency. Notably, 4 of the 5 genes upregulated by the high 5.0 μg/ml dose of CYR61 were relevant to immune cell recruitment and activation (CCL2, CXCL6, CXCL1, and IL32). Therefore, CYR61 appears to mildly activate human endothelial expression of genes relevant to immune cell recruitment.

CYR61 Strongly Activates Monocyte & Macrophage Gene Expression Relevant to Inflammation and Angiogenesis

Previous studies have shown that monocyte-derived macrophages are an abundant immune cell localized to CNV lesions. Consistent with this, there were many CCR2+ monocyte-derived macrophages in close proximity to CYR61 expressed by endothelial cells in choroidal neovascular lesions (see e.g., FIG. 12). Therefore, it was determined that the CYR61 produced by endothelial cells in CNV lesions could signal to and recruit or activate monocyte-derived macrophages. The transcriptional effect of CYR61 was first tested on human THP1 monocytes at 3 concentrations: 0.05, 0.5, or 5.00 μg/ml CYR61 (24 hr, n=5/group). On average, 35.3 million reads were obtained per sample of which 35.0 million (99.4%) mapped to the human genome. DEG analyses were performed to compare control vs. 0.05 μg/ml CYR61; control vs. 0.5 μg/ml CYR61; and control vs. 5.0 μg/ml CYR61. Similar to in HUVECs, there was a dose-dependent effect on the number of genes dysregulated with FDR<0.05: there were 0 DEGs for 0.05 μg/ml, 62 DEGs for 0.50 μg/ml, and 2,314 DEGs for 5.0 μg/ml. The most dysregulated genes were identified in monocytes treated with 5.0 μg/ml CYR61 with FDR<0.05 and |log 2(fold-change)|>1.5. There were 38 such genes and they were all upregulated (see e.g., FIG. 15B). Of note, 12 of these genes were also upregulated to a lesser degree at the lower 0.5 μg/ml CYR61 concentration. Many of the monocyte genes whose expression was activated by CYR61 are related to inflammation and angiogenesis including MMP9, IL411, ICAM1, C3, TNFAIP3, TLR8, SLAMF7, EBI3, TNFRSF9, FCAR, BCL3, C3AR1, CD83, CXCL8, IL1B, IL7R, CXCL1, and GPR84 (see e.g., FIG. 15B). Since monocytes differentiate into tissue macrophages, the effects of CYR61 on differentiated macrophages were tested. Bone marrow-derived macrophages (BMDMs) were prepared from wild type C57BL/6J mice, treated with a moderate dose of CYR61 (0.5 μg/ml) for 24 hr, and assessed for gene expression by RNA sequencing. An average of 61.2 million paired-end reads was obtained per sample (n=7 controls, n=4 CYR61-treated), and of these, an average of 58.0 million (94.8%) mapped to the mouse genome. There were 1,732 DEGs with FDR<0.05 when BMDMs were treated with 0.5 μg/ml CYR61. To broadly identify whether CYR61-responsive genes in BMDMs suggested perturbations in specific pathways which may provide insight into the mechanism by which CYR61 activates macrophages towards a potentially AMD-relevant state, pathway analysis was performed. Enrichr was queried using the 1,110 genes upregulated by CYR61 with FDR<0.05 for enrichment in the following gene sets: OMIM Disease and WikiPathway 2021 Human (see e.g., FIG. 15C-FIG. 15D). Genes upregulated by CYR61 were characteristic of “cholesterol metabolism”, “cholesterol biosynthesis pathway”, “VEGFA-VEGFR2 signaling pathway”, and “macular degeneration” (see e.g., FIG. 15C-FIG. 15D). Notably, perturbation of the VEGFA-VEGFR2 signaling pathway is of potential clinical relevance because this is currently the first-line target for therapy in the clinical management of neovascular AMD.

To identify the top genes responsive to CYR61 in macrophages, any genes with |log₂FC|>1.5 and FDR<0.05 in the RNA sequencing data were identified. There were 17 such genes and they were all upregulated (see e.g., FIG. 15E). In general, many of these genes were related to: inflammation and angiogenesis (Saa3, Mmp9/14, Cfb, Marco, Hp, Acod1, C3, Socs3, Ccl5). Some of these genes were also upregulated in THP1 human monocytes treated with CYR61 including Mmp9 and C3 (see e.g., FIG. 15B and FIG. 15E), To assess the activation state of macrophages treated with CYR61, the gene expression of 3 pro-inflammatory and 3 pro-angiogenic genes was tested by qPCR (see e.g., FIG. 15F). There was statistically significant upregulation of pro-inflammatory/anti-angiogenic markers such as 1b (↓2.6×) and Socs3 (↓3.4×), whereas pro-angiogenic markers such as Vegfa, Vcam1, and Icam1 were not changed (see e.g., FIG. 15F). Based on the macrophage activation markers tested, CYR61 appears to activate macrophages towards a pro-inflammatory state that has been characterized previously as anti-angiogenic.

CYR61-Activated Macrophages can Signal to Endothelial Cells

To test whether CYR61-activated macrophages can signal to endothelial cells, cells were cultured in transwell plates with a 0.4 μm pore size that allows secreted proteins to pass between the upper and lower compartments but restricts cell migration. HUVECs were plated in the bottom compartment, and the top compartment contained either: 1) cell-free control; 2) naïve BMDMs; or 3) CYR61-activated BMDMs (0.5 μg/ml, 24 hr). After 24 hr co-incubation, performed RNA sequencing was performed on the HUVECs. An average of 65.5 million reads per sample were obtained and of these, an average of 65.3 million reads (99.7%) mapped to the human genome. To identify any effects of naïve macrophages on HUVECs, DEG analysis was performed to compare HUVECs alone vs. HUVECs co-incubated with naïve BMDMs. In this analysis, there were only 2 dysregulated genes with FDR<0.05, suggesting that naïve macrophages do not influence HUVEC transcriptome via paracrine signaling. To identify paracrine macrophage effects on HUVECs activated by CYR61, DEG analysis was performed to compare HUVECS co-incubated with naïve macrophages vs. HUVECs co-incubated with CYR61-activated macrophages. There were 3,658 dysregulated genes with FDR<0.05. This indicates that while naïve macrophages do not produce abundant secreted factors that can signal to endothelial cells, CYR61-activated macrophages do. To identify which macrophage-to-endothelial signaling pathways are activated by CYR61, a database of 3,251 known ligand-receptor interactions was accessed. Interactions were identified for which the ligand was dysregulated in BMDMs treated with CYR61 (with FDR<0.05) and the receptor was dysregulated in HUVECs co-incubated with CYR61-activated BMDMs compared to coincubation with naïve BMDMs (with FDR<0.05). There were 58 such interactions (see e.g., FIG. 15G-FIG. 15H), including many angiogenesis-modifying signaling pathways such as VEGFA, THBS1, MMP9, and ANGPTL2 (see e.g., FIG. 15G). Some pro-angiogenic molecules such as Angptl2 and Vegfa are downregulated in BMDMs treated with CYR61 whereas the anti-angiogenic molecule Thbs1 was upregulated (see e.g., FIG. 151). Mmp9, a pro-angiogenic molecule, is upregulated (see e.g., FIG. 151). Therefore, CYR61 activates a mixed pattern of angiogenesis-related macrophage-to-endothelial signaling pathways.

Macrophage Genes Responsive to CYR61 are Upregulated in Choroidal Macrophages

RNA sequencing showed that there were genes that were responsive to CYR61 treatment in murine BMDMs with log 2(foldchange)|>1.5. Since CYR61 is expressed by choroidal endothelial cells and may signal to local macrophages, gene expression of CYR61-responsive genes was assessed in the human scRNAseq dataset by comparing macrophages localized in the choroid to monocytes in the peripheral blood. Of the 17 CYR61-responsive genes in murine BMDMs, 12 paired with a single human ortholog and 5 had no ortholog (Saa3, Gm14493, Gm49383, Rps12I1, Nxpe5).

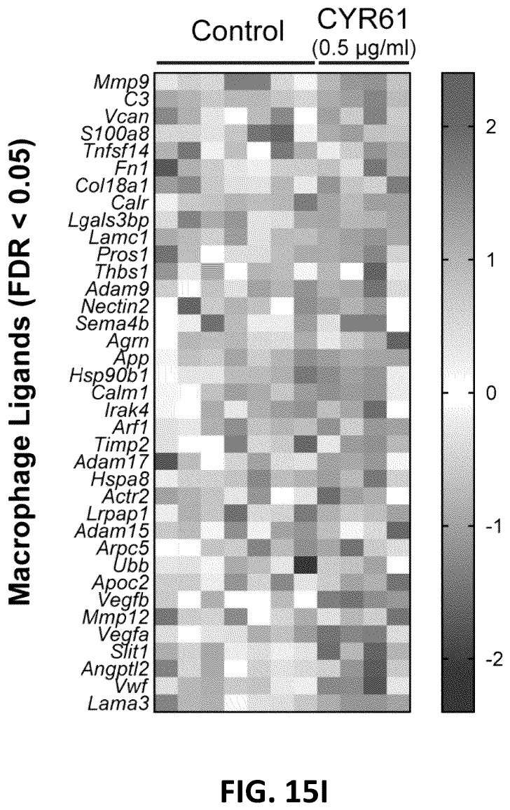

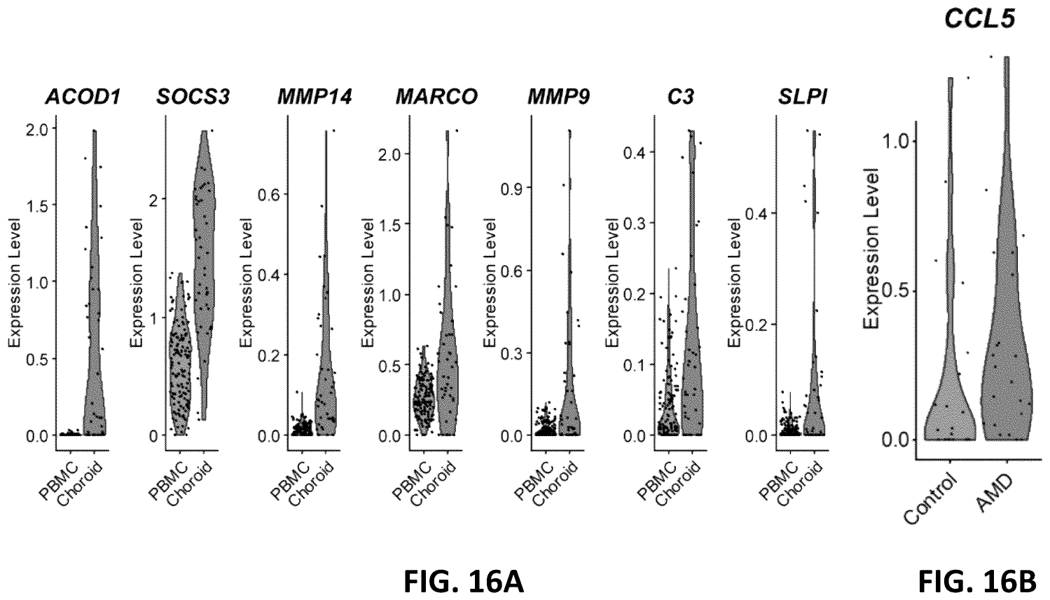

Of the 12 CYR61-activated macrophage genes with a human ortholog, 7 were also upregulated (pseudobulked unadjusted p value <0.05) in choroidal macrophages compared to circulating monocytes (see e.g., FIG. 16A). These were: SOCS3, ACOD1, MARCO, MMP9, MMP14, SLPI, C3 (see e.g., FIG. 16A). Therefore, CYR61 produced by choroidal endothelial cells could contribute in part to the unique gene expression profile of macrophages localized to the choroidal microenvironment.

Since CYR61 was upregulated in choroidal veins in AMD, gene expression of the 12 top CYR61-responsive genes with human orthologs was also assessed in choroidal macrophages comparing control vs. AMD. CCL5 was upregulated in AMD choroidal macrophages (pseudobulked unadjusted p-value <0.05), consistent with upregulation of Cc/5 when BMDMs are treated with CYR61 (see e.g., FIG. 16B).

CYR61 Inhibits Pathologic Ocular Angiogenesis

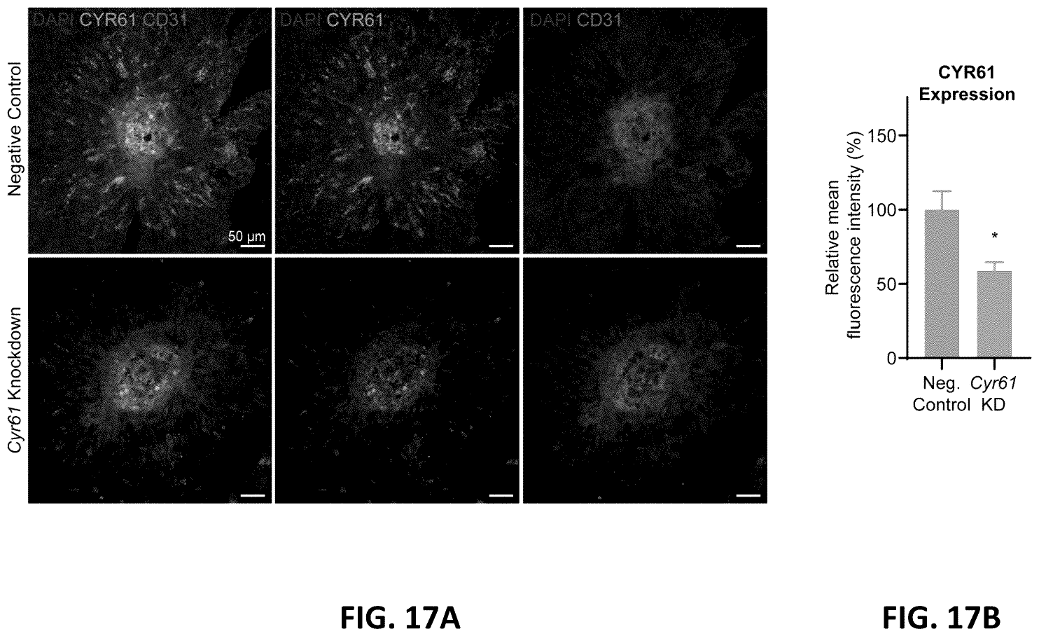

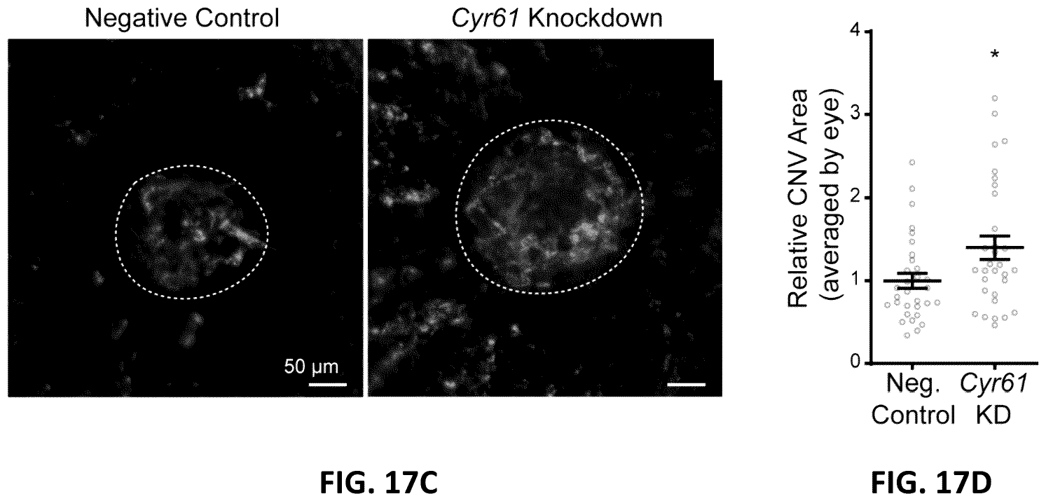

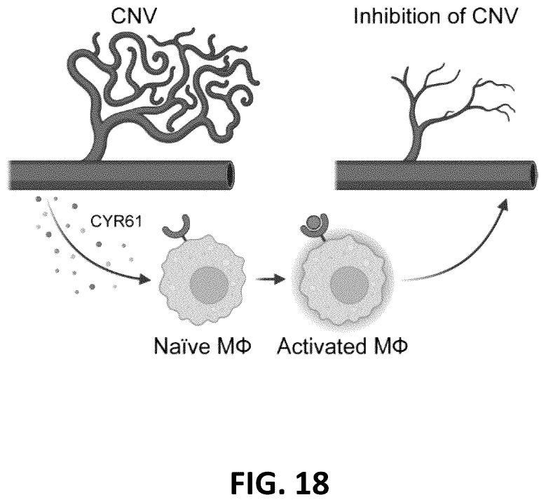

To test the role of CYR61 in pathologic ocular angiogenesis, a locked nucleic acid was designed that targets Cyr61 transcript. CNV was induced in mice by laser injury immediately followed by intravitreal administration of either negative control or anti-Cyr61 locked nucleic acid. CNV lesions were assessed 7 days later. The locked nucleic acid inhibited Cyr61 expression in mouse CNV lesions by ˜41% (see e.g., FIG. 17A-FIG. 17B). When CNV severity was compared, the lesion size was significantly increased in the mice treated with anti-Cyr61 locked nucleic acid by ˜40% (see e.g., FIG. 17C-FIG. 17D). Therefore, CYR61 inhibits pathologic ocular angiogenesis in this mouse model of CNV (see e.g., FIG. 18).

Discussion

As described herein, scRNAseq data of choroid and circulating peripheral immune cells in AMD was utilized to: i) characterize the transcriptional signatures that distinguish immune cells in the choroid from those circulating in the peripheral blood; ii) better understand the potential molecular signaling pathways between choroid and peripheral immune cells; and iii) identify novel therapeutic targets for the treatment of this debilitating disease. First, when compared to peripheral blood immune cells, those local to the choroid had gene expression profiles that were enriched for inflammation, chemokine signaling, vessel disease, lipid response, and neuro- & macular degeneration. Second, the cross-tissue ligand-receptor interaction analysis highlighted several genes that are known to be important in AMD, including VEGF receptor 2, the first line target in the clinical management of neovascular AMD. The analysis also identified APOE whose variants are associated with AMD, along with THBS1 and LGALS1 which have been shown to inhibit or promote CNV in mouse models of disease. Previous work was validated by showing that genetic deletion of Thbs1 increased CNV severity in mice, highlighting the potential specificity of this multi-tissue approach in identifying high-value interactions relevant to disease pathogenesis. Third, CYR61 was identified as a potentially druggable target that inhibits pathologic ocular angiogenesis.

It may be difficult to ascertain the pathogenic relevance of the direction of ligand or receptor dysregulation in the interpretation of the network of interactions dysregulated in AMD. For instance, VEGF receptor 2 activates a pro-angiogenic pathway, yet it is downregulated in choroidal arteries in AMD. The contradictory finding of a downregulated, disease-promoting receptor in AMD patients could be potentially due to: only 2 of the 11 patients presenting with neovascular AMD in the choroid dataset; compensation from negative feedback; clinical therapy targeting this pathway; a combination of these factors or other as yet unknown reasons. Similarly, CNV-promoting LGALS1 was downregulated in AMD choriocapillaris whereas CNV-inhibiting THBS1 was upregulated in AMD arteries. Moreover, the network includes downregulation of APOE in SMCs. Though APOE has isoform-specific associations with AMD risk, it is not yet clear how these genetic variations relate to APOE expression in specific tissues and cell types. The unintuitive or uninterpretable direction of dysregulation of some ligand-receptor interactions in the context of disease could reflect diverse molecular roles in disease onset vs. disease progression, therapy effect, disease chronicity or severity, spectrum of disease presentation (i.e. early vs. wet vs. dry), compensation from negative feedback, or a combination of these and other factors involved in this complex aging disease where genetics, age, and lifestyle contribute to disease pathogenesis. Therefore, novel disease-relevant signaling pathways identified by ligand-receptor analysis should be validated for functional consequence.

Mechanistic studies were performed herein of CYR61 as a ligand of interest that is expressed by human choroidal endothelial cells in AMD and in murine CNV lesions. Notably, a network was delineated of macrophage-to-endothelial cell signaling pathways that are perturbed by CYR61 treatment, including many angiogenesis-modulating pathways. When ocular Cyr61 expression was knocked down, CNV size increased, consistent with the finding that CYR61 activates macrophages towards a pro-inflammatory, anti-angiogenic state. Taken together, the data suggest that in this context of laser injury-induced CNV, activation of CYR61 expression could be a protective response that inhibits pathologic angiogenesis.

This finding of CYR61 as an anti-angiogenic molecule is surprising given its known pro-angiogenic roles during development and in other tissue contexts. Germline deficiency of Cyr61 leads to premature embryonic death due to impaired vascularization of the placenta as well as other vascular defects. In adult rabbits, CYR61 promoted re-vascularization of the hindlimb following excision of the femoral artery. Over-expression of CYR61 in gastric adenocarcinoma cells enhanced their tumorigenicity and increased the vascularity of tumors. In the eye, CYR61 was shown to promote corneal neovascularization in rats. Though CYR61 appears to promote angiogenesis in many contexts, there is evidence that CYR61 may inhibit angiogenesis in certain cases. For instance, in studies of murine oxygen-induced retinopathy, Hasan et al. reported that over-expression of Cyr61 reduced the formation of pathologic neovessels (see e.g., Hasan et al. (2011) J Biol Chem. 286, pp. 9542-9554). However, this is contradictory to reports from two other research groups that antibody/siRNA blockade of CYR61/Cyr61 reduced pathologic retinal neovascularization in oxygen-induced retinopathy. Similarly, though it is reported herein that CYR61 inhibits CNV, Sun et al. reported that CYR61 promotes CNV in diabetic mice (see e.g., Sun et al. (2017) Sci Rep. 7, 14925). The divergent pro- or anti-angiogenic roles of CYR61 could depend on the microenvironment, experimental conditions, context, cell types, and/or cell surface receptors involved. In the CNV model used in the present disclosure, CYR61 may regulate angiogenesis at least in part due to the critical involvement of immune cells-specifically macrophages-in the pathologic process, reminiscent of human disease. In this context, CYR61 may activate macrophages towards a pro-inflammatory, anti-angiogenic phenotype that has been described to inhibit CNV. This is consistent with CYR61's known capability to polarize monocytes/macrophages towards the pro-inflammatory M1 phenotype via NF-KB. Characterization will include the functional and mechanistic role of CYR61 in human AMD.

Consistent with previous work, this work also highlights aberrant lipid metabolism as a central feature of ocular immune cells in AMD. For instance, in comparing choroidal vs. peripheral immune cells, dysregulation of genes related to “lipid and atherosclerosis” was identified. Additionally, the network of choroid-peripheral immune cell interactions dysregulated in AMD included the apolipoprotein APOE and the lipoprotein lipase transporter GPIHBP1. Of mechanistic relevance, when BMDMs were treated with CYR61, there was dysregulation of genes related to “cholesterol metabolism” and “cholesterol biosynthesis”. Thus, CYR61 not only may regulate pathologic angiogenesis, but also may contribute to lipid dyshomeostasis in ocular immune cells and play a role in the accumulation of lipid-rich ocular deposits-drusen-in AMD.

Of conceptual importance, peripheral immune cells exhibited strikingly few transcriptional changes in AMD, especially in comparison to the AMD-associated changes in cells localized to the choroid. The paucity of AMD-associated changes in circulating immune cells is somewhat expected, as AMD is an ocular disease that locally affects the eye and is thus unlikely to exert substantial effects on the bulk population of circulating peripheral immune cells. Despite their apparent transcriptional insensitivity in AMD, peripheral immune cells invade the eye during the pathogenesis of CNV. A model is proposed herein by which specific choroidal ligands may engage with receptors on peripheral immune cells passing through the ocular choroidal vasculature. These signaling interactions may recruit peripheral immune cells to enter the eye or influence their in situ activation to regulate or promote disease initiation and progression.

Future work may investigate the role of tissue resident microglia. Microglia are the predominant resident immune cell in the retina and perform important sentinel functions during homeostasis. These cells are highly plastic and the microenvironmental and activation cues determine the roles they play in disease processes. Multiple studies have shown that microglia when activated in animal models of retinal disease can promote or protect against retinal neurodegeneration. In neovascular AMD, the immune privilege within the sub-retinal space is abrogated as neovascular sprouts grow from the choroid through the Bruch's membrane into the sub-retinal space to form CNV. In CNV, monocyte-derived macrophages play a dominant role in animal models of injury induced CNV and are the most common immune cell type in human CNV secondary to AMD. Nonetheless, tissue resident microglia may likely play important roles in cell-to-cell signaling in AMD and their role warrants further investigation.

Overall, the present disclosure includes a proof-of-concept highlighting the enormous potential for using multi-tissue datasets to screen for cross-tissue signaling pathways that may be targeted by novel therapies to treat human disease. The analytical approach described is potentially applicable for a myriad conditions characterized by inappropriate immune cell invasion of peripheral tissues, especially of the central nervous system. The present disclosure utilizes scRNAseq data analysis and, in some embodiments, may be complemented by bulk cellular or tissue RNA sequencing data, protein quantifications of ligands in tissues or fluids, flow cytometry analysis of cell-surface receptors, or other laboratory methods depending on the disease context under investigation, tissue accessibility, and availability of resources and laboratory techniques. This novel screening platform was applied to identify CYR61 as a regulator of pathologic ocular angiogenesis. With relevance to human disease as disclosed herein, some embodiments employ CYR61's anti-angiogenic properties to augment first line anti-VEGF therapies in the management of neovascular AMD.

Methods

Patient Classification and Isolation of Patient PBMCs

Samples were classified as coming from patients with no AMD; early AMD; or AMD with geographic atrophy and/or CNV based on established clinical criteria. Early AMD patients had either small or moderate drusen (<125 μm) or pigment changes in at least 1 eye but no CNV or geographic atrophy in either eye. Patients with geographic atrophy and/or CNV in at least 1 eye were labeled as such. Any patients receiving immunotherapy, chemotherapy, or radiation for underlying autoimmune/immune-mediated diseases or cancers were excluded as this could affect circulating immune cells. Blood was collected from patients by venous blood draw into K₂EDTA coated BD Vacutainer Venous Blood Collection Tubes. To purify PBMCs, the blood was centrifuged, the buffy coat was isolated, and red blood cells lysed, leaving nucleated PBMCs behind. The PBMCs were stored in liquid nitrogen for long-term storage.

Single Cell RNA Sequencing of PBMCs

Cells were quickly thawed following retrieval from liquid nitrogen storage, washed twice in Dulbecco's Modified Eagle Medium (Gibco), and treated with 200 KU/ml DNasel (Sigma). Live and dead cells were counted using a dye exclusion assay with a Tecan automated counter. The total cell concentration was adjusted to 700-1200 cells/μl to be used for the 10× single cell gene expression pipeline. Single cell RNA sequencing was performed on the microfluidic-based 10s Genomics platform according to manufacturer's instructions. The Chromium Single Cell 3′v3.1 Reagent Kit was used. Briefly, cells were partitioned into individual droplets that contain a barcoded gel bead using the 10× Genomics Chromium Controller. The cells were then lysed and reverse transcribed RNA to cDNA. The gel bead emulsion was broken, cDNA amplified and fragmented, and Illumina adapters added. Samples were sequenced on the Illumina NovaSeq 6000 platform.

Single Cell RNA Sequencing Analysis

Import, quality control, and cell type annotation of PBMC dataset. The raw FASTQ sequencing files were processed using CellRanger 4.0.0 with alignment to the 10× Genomics human reference genome (refdata-gex-GRCh38-2020-A). The filtered count matrices were imported into Seurat v454, and each cell was assigned a unique identifier to prevent overlap of barcodes between different samples. The count matrices were normalized, log-transformed, and scaled to remove unwanted sources of variation such as discrepancies in sequencing depth. The top 2000 highly variable genes were identified for principal component analysis. Integration across samples was performed to account for any sample- or experiment-specific batch effects using the Harmony package. These harmony embeddings were used to run Uniform Manifold Approximation and Projection dimensional reduction to 2 dimensions. Cells were clustered according to the Louvain algorithm and marker genes identified for each cluster using Wilcoxon rank sum tests comparing each cluster to all other clusters. The manual annotation of cell types was validated as consistent with automated annotation assigned using Azimuth using the “pbmcref” reference.

Import and integration of choroid dataset with PBMC dataset. The choroid scRNAseq dataset was downloaded from the Gene Expression Omnibus (accession: GSE183320). The normalized count matrix was converted to the raw count matrix using the inverse of Seurat's NormalizeDatao function executed using the default parameters. The raw count matrix was imported into Seurat v4 to be processed using the same functions and parameters as described above for the PBMC dataset. The original cell types annotated by Voigt et al. were maintained for the choroidal cell types. The choroid and PBMC datasets were merged and integrated across all of the samples using the Harmony package.

Differential gene expression comparing choroidal to circulating immune cells. To identify unique gene signatures of choroidal vs. circulating immune cells, gene expression of each cell type was first pseudobulked by patient. Then, the following comparisons were run using Seurat's FindMarkerso function: monocyte/macrophages (macrophages in choroid vs. monocytes in circulation); B cells (in choroid vs. circulation); T cells (in choroid vs. in circulation); and dendritic cells (dendritic cells in choroid vs. cDCs in circulation). Differentially expressed genes were those with adjusted p-values <0.05 and |log₂FC|>0.5.

Differential gene expression analysis of control vs AMD. To identify differentially expressed genes for each cell type comparing control to AMD patients, a pseudobulked approach was used where gene expression of all cells of a particular cell type are averaged by patient. Seurat's differential gene expression analysis with the FindMarkerso function was designed for sample sizes of 100-1000s of cells and uses the conservative Bonferroni correction. For the pseudobulked comparisons of 10s of patients, a more sensitive approach was used by using the unadjusted p-values to determine statistical significance since further hits were validated in mouse models of disease. The usage of unadjusted p-values for pseudobulked scRNAseq datasets has been used previously. Differentially expressed genes were those with unadjusted p-values <0.05 and |log₂FC|>0.5.

Ligand-receptor analysis. The package SingleCellSignalR was used on the pseudobulked scRNAseq dataset with the function cell_signaling( ) and the following parameters: int.type=“paracrine”, s.score=0.5, and tol=0.05. To narrow the full intercellular signaling network to those that may be relevant to AMD, 3 sets of filters were applied. First, only interactions for which either the ligand and/or receptor was dysregulated in AMD in the appropriate cell type were included. Second, any interactions involving platelets, red blood cells, Schwann cells, rod/RPE cells, melanocytes, or any immune cells in the choroid were excluded. Finally, only interactions between cells of the choroid and PBMCs were included. Flow graphs were generated using the alluvial package and graphs of intercellular networks were generated using Cytoscape.

Mice

2-5 month old C57BL/6J and Thbs1−/− (strain 006141) mice from Jackson Laboratories were used. To identify CCR2+ monocyte-derived macrophages in mouse choroidal neovascular lesions, heterozygote Ccr2+/gfp mice available from Jackson Laboratories (strain 027619) were used. Mice were housed in a barrier facility with a 12:12 light/dark cycle with water and food provided ad libitum.

Laser Injury-Induced Choroidal Neovascularization in Mice

Laser injury. CNV was induced in mice using a laser injury-induced model. Mice were anesthetized using a cocktail of ketamine and xylazine and dilated pupils using topical tropicamide. A Phoenix MICRON Image-guided laser system equipped with a green 532 nm Merilas 532a laser was used with the following settings: 50 μm spot size, single pulse of 70 ms pulse length, 800 mW power. After laser injury, a topical antibiotic ointment was applied at the ocular surface, mice were kept on a heating pad and monitored until they awoke from anesthesia. Mice were checked daily and sacrificed on day 7 after laser injury.

Preparation of RPE/choroid/sclera complexes. For analysis of whole-mounted RPE/choroid/sclera, mice were deeply anesthetized and perfused through the heart with 10 ml ice-cold PBS if eyes were to be used for immunohistochemistry. If eyes were to be used for CNV lesion size measurement, 100 ul of FITC-dextran (50 mg/ml; 2,000,000 MW; Sigma) was injected into the femoral vein and allowed to circulate for 5 min. After sacrifice, eyes were enucleated s using forceps, rinsed 1× in ice-cold PBS to remove residual blood and fur, and then fixed in 4% paraformaldehyde or 10% neutral buffered formalin for 2-6 hr at room temperature. The eyes were then hemisected ˜2 mm posterior to the limbus and the neurosensory retina removed from the posterior eye cup. These RPE/choroid/sclera complexes were either used for immunostaining as described below (“Immunostaining”) or immediately mounted for CNV lesion size quantifications. To flatten the tissue on a slide, ≥4 radial incisions spaced equally around the RPE/choroid/sclera complex were used, taking care to avoid cutting through CNV lesions. To quantify CNV lesion sizes, z-stack images of lesions were taken using a Leica DMi8 microscope and lesion sizes quantified using Metamorph as previously described.

Eye sections. If eyes were to be used for sectioning, mice were deeply anesthetized and perfused through the heart with 10 ml ice-cold PBS. Eyes were enucleated with forceps and immediately embedded in Tissue-Tek OCT media (Sakura) to be frozen using dry ice. A Leica CM1950 cryostat was used to cut fresh frozen 15 μm eye sections and tissue sections were deposited onto positively charged glass slides to be stored at −80° C.

RNAscope

mRNA localization of Cyr61 was assessed in fresh frozen tissues sections using the RNAscope Multiplex Fluorescent Reagent Kit v2 (ACDBio) according to the manufacturer's protocol. Briefly, tissue sections were fixed using 10% neutral buffered formalin at 4° C. for 15 minutes. Slides were washed twice with PBS to remove excess fixative and then tissue sections were dehydrated using ethanol. Then tissue sections were treated with hydrogen peroxide for 10 minutes followed by protease IV treatment for 15 minutes. The tissue was hybridized with probes recognizing Cyr61 and Cd31 for 2 hours at 40° C. Afterwards, amplification steps 1-3 were performed and HRP-C1 and HRP-C2 signals developed. Opal 520 and Opal 620 in TSA buffer (1:1500) were used to visualize transcripts. Finally, the tissue sections were mounted using FluorSave mounting reagent (Millipore) and images acquired using a Zeiss LSM800 confocal microscope.

Immunostaining

Whole-mounted RPE/choroid/sclera. Protein localization of CYR61 and CD31 was assessed in whole-mounted RPE/choroid/sclera complexes. The fixed tissue was blocked for 2 hrs at room temperature using PBS containing 5% serum corresponding to the secondary antibody host and 0.3% Triton X-100. Tissues were incubated with primary antibodies overnight followed another overnight incubation with secondary antibodies, both at 4° C. The following primary antibodies were used: anti-CYR61 (Cell Signaling Technology 39382), anti-CD31 (Millipore MAB1398), and anti-CD45 (BD Biosciences 550539). Following the staining procedure, tissue was mounted by making ≥4 radial incisions to enable flattening of tissue and then acquired images on a Zeiss LSM800 confocal microscope.

Fresh frozen tissue sections. For immunostaining of tissue sections, fresh frozen tissue sections were fixed using 10% neutral buffered formalin for 1 hour at room temperature. Residual fixative was washed away with PBS prior to blocking using PBS containing 5% serum corresponding to the secondary antibody host and 0.3% Triton X-100. Tissue sections were incubated with primary antibodies overnight at 4° C. followed by incubation with secondary antibodies for 1 hour at room temperature. The following antibodies were used: anti-CYR61 (Cell Signaling Technology 39382) and anti-CD31 (Millipore MAB1398). Following the staining procedure, coverslips were applied and images were acquired using a Zeiss LSM800 confocal microscope.

Cells

Murine bone marrow-derived macrophages. BMDMs were prepared from C57BL/6J mice following previously described methods. Briefly, mice were euthanized and bone marrow collected from both tibias and femurs. Bone marrow cells were resuspended in BMDM differentiation media consisting of Dulbecco's Modified Eagle Medium supplemented with 10% FBS, 10% CMG-conditioned media, 1% penicillin/streptomycin, and 1% L-glutamine. Cells were allowed to differentiate into macrophages for 5-6 days with media replenished on day 3. After differentiation, BMDMs were replated in BMDM mature media consisting of DMEM with 10% FBS, 2% CMG-conditioned media, 1% penicillin/streptomycin, and 1% L-glutamine.

Human umbilical vein endothelial cells. Primary HUVECs were purchased from ATCC (PCS-100-010) and maintained following manufacturers recommendations.

THP1 human monocytes. Cells (ATCC TIB-202) were obtained from Dr. Todd A. Fehniger (Washington University School of Medicine in St. Louis). THP1 cells were maintained in RPMI 1640 media containing 1% penicillin/streptomycin, 1% L-glutamine, and 10% FBS.

RNA Sequencing of HUVECs Treated with CYR61

HUVECs were incubated with recombinant human CYR61 (PeproTech 120-25) at 4 concentrations: 0.00, 0.05, 0.50, and 5.00 μg/ml for 24 hr (n=5 wells/group). After incubation, cells were washed twice with PBS, then TRIzol (Ambion) and RNeasy Plus Mini Kit (Qiagen) were used to extract total RNA following the manufacturer's recommended protocol. The quality and quantity of the RNA samples was quantified with an Agilent Bioanalyzer or 4200 Tapestation. All 20 samples had RIN≥9.7. ds-cDNA was prepared using the SMARTer Ultra Low RNA kit for Illumina Sequencing (Takara-Clontech) following the manufacturer's protocol. Then, cDNA was fragmented, blunt ended, an A base was added to the 3′ ends, and then Illumina sequencing adapters were ligated to the ends. The ligated fragments were amplified and unique dual index tags were incorporated. The fragments were then sequenced on an Illumina NovaSeq 6000 using paired end reads extending 150 bases at the McDonnell Genome Institute at Washington University School of Medicine in St. Louis. Sequenced reads were then aligned and quantitated to the Ensembl release 101 primary assembly with an Illumina DRAGEN Bio-IT on-premise server running version 3.9.3-8 software.

RNA Sequencing of THP1 Cells Treated with CYR61

THP1 cells were incubated with recombinant human CYR61 (PeproTech 120-25) at 4 concentrations: 0.00, 0.05, 0.50, and 5.00 μg/ml for 24 hr (n=5 wells/group). After incubation, cells were washed twice with PBS, then TRIzol (Ambion) and RNeasy Plus Mini Kit (Qiagen) were used to extract total RNA following the manufacturer's recommended protocol. The quality and quantity of the RNA samples was then quantified with an Agilent Bioanalyzer or 4200 Tapestation. All 20 samples had RIN=10.0. Using 500 ng-1 ug of starting total RNA, ribosomal RNA was removed using an RNase-H method using RiboErase kits (Kapa Biosystems). mRNA was fragmented in reverse transcriptase buffer and samples heated to 94 degrees for 8 minutes. mRNA was reverse transcribed to cDNA using SuperScript III RT enzyme (Life Technologies, per manufacturer's instructions) and random hexamers. A second strand reaction was performed to yield ds-cDNA. The cDNA was blunt ended, an A base added to the 3′ ends, and then Illumina sequencing adapters were ligated to the ends. The ligated fragments were amplified for 12-15 cycles using primers incorporating unique dual index tags. Fragments were sequenced on an Illumina NovaSeq 6000 using paired end reads extending 150 bases. Basecalls and demultiplexing was performed with Illumina's bcl2fastq software with a maximum of one mismatch in the indexing read. Sequenced reads were aligned to the Ensembl release 101 primary assembly with STAR version 2.7.9α₁, and then gene counts derived from the number of uniquely aligned unambiguous reads by Subread:featureCount version 2.0.32. Expression of known Ensembl transcripts was quantified using Salmon version 1.5.23. Sequencing performance was assessed by the total number of aligned reads, total number of uniquely aligned reads, and features detected. RSeQC version 4.04 was used to quantify the ribosomal fraction, known junction saturation, and read distribution over known gene models. Finally, standard differential gene expression analysis of gene-level features was performed using EdgeR and Limma.

Gene Expression Studies of BMDMs Treated with CYR61

BMDM treatment and RNA isolation. BMDMs were incubated with recombinant human CYR61 (PeproTech 120-25) in BMDM mature media at 2 concentrations: 0.00 and 0.50 μg/ml for 24 hr (n=8 control wells, n=4 CYR61-treated wells). After incubation, cells were washed twice with PBS, then TRIzol (Ambion) and RNeasy Plus Mini Kit (Qiagen) were used to extract total RNA following the manufacturer's recommended protocol. The quality and quantity of the RNA samples was quantified with an Agilent Bioanalyzer or 4200 Tapestation. All 12 samples had RIN 9.5.

RNA sequencing. For RNA sequencing experiences, ribosomal RNA was first removed by an RNase-H method using RiboErase kits (Kapa Biosystems). Then, mRNA was fragmented and reverse transcribed to cDNA using SuperScript III RT enzyme (Life Technologies) and random hexamers following the manufacturer's instructions. A second strand reaction was performed to yield ds-cDNA. Then, the cDNA was blunt-ended, an A base was added to the 3′ ends, and then Illumina sequencing adapters were ligated to the ends. Ligated fragments were amplified and unique dual index tags were incorporated.

Fragments were sequenced on an Illumina NovaSeq 6000 using paired end reads extending 150 bases at the McDonnell Genome Institute at Washington University School of Medicine in St. Louis. RNA sequencing reads were then aligned and quantitated to the Ensembl release 101 primary assembly with an Illumina DRAGEN Bio-IT on premise server running version 3.9.3-8 software. 1 of the 8 control samples was identified to be an outlier and excluded\from further analyses.

qPCR. For qPCR assessments of Il1b, Socs3, Nos2, Icam1, Vcam1, and Vegfa expression, total RNA was reverse transcribed to cDNA using the High Capacity cDNA Reverse Transcription Kit (Applied Biosystems). Target gene expression was normalized to geometric mean of the following housekeeping genes: 18S, Actb, Gapdh, and Hprt. Gene expression differences were calculated using the ΔΔCt method.

RNA Sequencing of HUVECs Co-Incubated with BMDMs

HUVECs were plated in the bottom compartments of 24-well transwell plates (0.4 μm pore size, Corning) along with the following in the top compartments: 1) cell-free (n=6 wells); 2) naïve BMDMs (n=6 wells); or 3) CYR61-treated BMDMs (0.5 μg/ml, 24 hr, n=6 wells). After coincubation for 24 hrs, HUVECs were washed twice with PBS, then TRIzol (Ambion) and RNeasy Plus Mini Kit (Qiagen) were used to extract total RNA following the manufacturer's recommended protocol. The quality and quantity of the RNA samples were quantified with an Agilent Bioanalyzer or 4200 Tapestation. All 18 samples had RIN≥8.9. ds-cDNA was prepared using the SMARTer Ultra Low RNA kit for Illumina Sequencing (Takara-Clontech) per manufacturer's protocol. Then, cDNA was fragmented, blunt ended, an A base was added to the 3′ ends, and then Illumina sequencing adapters were ligated to the ends. The ligated fragments were amplified and unique dual index tags were incorporated. Then the fragments were sequenced on an Illumina NovaSeq 6000 using paired end reads extending 150 bases at the McDonnell Genome Institute at Washington University School of Medicine in St. Louis. RNA-seq reads were then aligned and quantitated to the Ensembl release 101 primary assembly with an Illumina DRAGEN Bio-IT on-premise server running version 3.9.3-8 software.

Knockdown of Cyr61 in Mouse CNV Lesions

Design. The mouse transcript sequence NM_010516.2 was used to design a locked nucleic acid targeting Cyr61 in Qiagen's GeneGlobe tool.

Validation. To validate that the designed locked nucleic acid could knockdown Cyr61 expression in murine CNV lesions, laser injury was induced (see “Laser injury-induced choroidal neovascularization”) and immediately after 2 μl of 150 μM negative control or anti-Cyr61 locked nucleic acid was injected into the vitreous using a 31G Hamilton syringe. After 7 days, the mice were sacrificed and CYR61 abundance assessed in whole-mounted tissue by immunostaining (see “Immunostaining”).

Assessments of CNV severity. To assess CNV lesion size, laser injury was induced and immediately after negative control or anti-Cyr61 locked nucleic acid was administered by intravitreal injection (2 μl, 150 μM). Seven days later, CNV lesion size was assessed by visualizing CNV lesion size using FITC-dextran to highlight vascular tissue (see “Laser injury-induced choroidal neovascularization”).

Statistics

The statistical analyses used for single cell RNA sequencing and bulk RNA sequencing are described above. For all other data, statistical analyses were performed using GraphPad Prism 9. The normality of the data was first assessed graphically and by using a Kolmogorov-Smirnov test, using nonparametric alternatives when appropriate. When applicable, outliers were identified and excluded using the ROUT method. When comparing a single variable between two different groups, 2-tailed t tests or 2-tailed Mann Whitney tests were used. For other analyses, statistical significance was assessed using the appropriate parametric or nonparametric test, as indicated in figure legends. A p-value <0.05 was considered statistically significant.

Study Approval

Generation of the PBMC scRNAseq dataset was approved by the Human Research Protection Office of Washington University School of Medicine in St. Louis. Written informed consent was obtained from all subjects prior to enrollment in the study. Experiments involving mice were approved by Washington University's Institutional Animal Care and Use Committee.

### Example 2: Cyr61-Treated Immune Cells as a Therapeutic for AMD

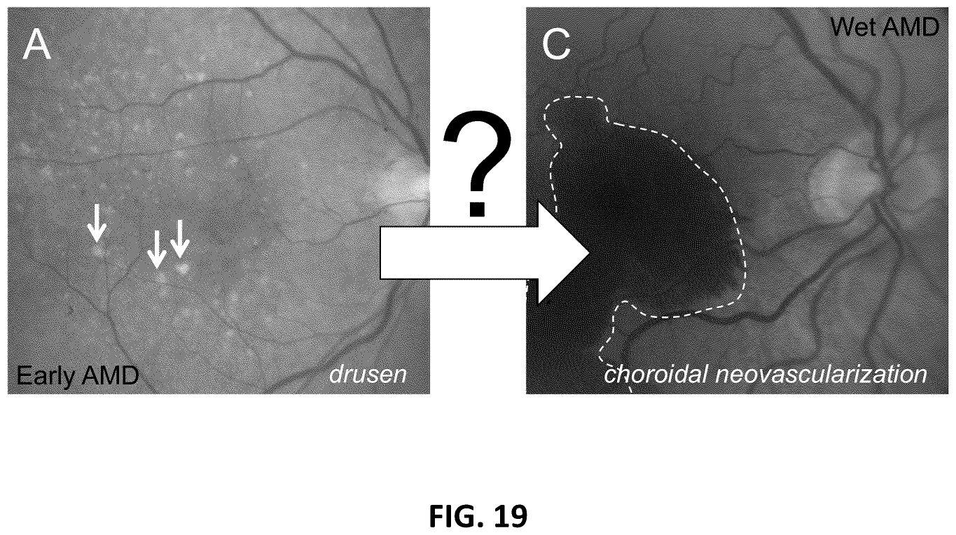

Age-related macular degeneration (AMD) is the leading cause of elderly blindness in industrialized nations. Disease onset involves lipid-rich deposits called drusen that form beneath the retina. Vision loss can occur slowly due to photoreceptor death and/or can occur suddenly due to leaky blood vessels that develop from the choroidal vascular bed that underlies and nourishes the retina (see e.g., FIG. 19). Pathologic evidence, genetic studies, and animal models of AMD suggest that immune cells, particularly monocyte-derived macrophages, are recruited to the choroid in AMD.

However, it remains unknown what cross-tissue signaling occurs in AMD that recruits/activates immune cells to the eye and/or promotes choroidal neovascularization (CNV). To assess for these disease-relevant cell-cell interactions, single cell RNA sequencing datasets of peripheral blood mononuclear cells (PBMCs) and of choroid were generated (see e.g., Example 1). CYR61 was identified as potentially playing a role in AMD (see e.g., Example 1).

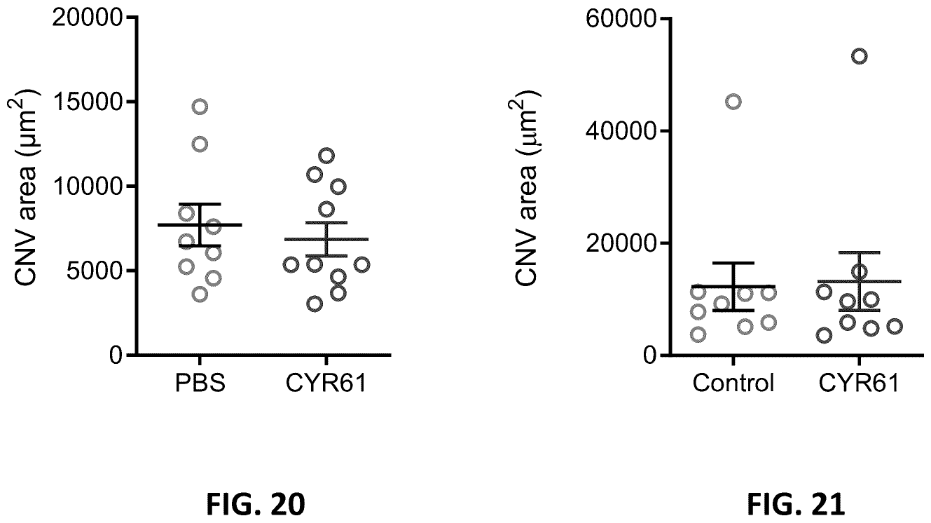

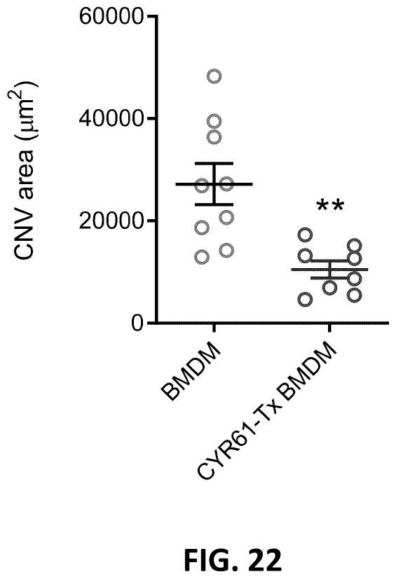

CYR61 is a secreted matricellular protein known to regulate angiogenesis in development and cancer, but its role in AMD is not well understood. As shown herein, in mice, CYR61 was strongly expressed in CD31+ endothelial cells in laser-induced choroidal neovascular (CNV) lesions which also contained CD45+ immune cells (see e.g., Example 1). In order to test the effect of Cyr61 on immune cells, murine bone marrow-derived macrophages (BMDMs) were incubated with CYR61 (0.5 ug/mL). The effects of intravitreal injections of CYR61, media from CYR61-treated BMDMs, or CYR61-treated BMDMS on laser-induced CNV in mice (n=8-10 eyes per group) were tested. Only CYR61-treated BMDMs inhibited CNV lesion size whereas there were no differences in the other groups (see e.g., FIG. 20, FIG. 21, and FIG. 22). These data indicate that CYR61 regulates ocular pathological angiogenesis in a macrophage-dependent manner. CYR61 and the other ligand/receptors identified may be novel opportunities for AMD therapy to support existing anti-VEGF strategies.

What is claimed is:

1. A method of treating ocular neovascularization in a subject, the method comprising:
administering a CYR61 activating agent to an immune cell to form a treated immune cell; and
administering the treated immune cell to the subject.

2. The method of claim 1, wherein the CYR61 activating agent is CYR61.

3. The method of claim 1, wherein the subject has macular degeneration, age-related macular degeneration (AMD), a retinal disease, or diabetic retinopathy.

4. The method of claim 1, wherein the immune cell is selected from a macrophage and a monocyte.

5. The method of claim 4, wherein the macrophage is selected from a monocyte-derived macrophage, a bone marrow-derived macrophage (BMDM), and a choroidal macrophage.

6. The method of claim 1, wherein the subject has at least one choroidal neovascular (CNV) lesion.

7. The method of claim 6, wherein administering the treated immune cell to the subject reduces CNV lesion size.

8. The method of claim 1, further comprising administering to the subject an anti-VEGF treatment.

9. The method of claim 1, wherein the treated immune cell is administered to the subject intravitreally.

10. The method of claim 1, wherein administering the CYR61 activating agent to the immune cell upregulates expression of one or more genes selected from the group consisting of Saa3, Mmp9, Gm14493, Cfb, Mmp14, Slpi, Gm49383, Marco, Rps12I1, Pilra, Hp, CcI5, Nxpe5, Acod1, C3, Socs3, Mid1-ps1, Il1b, and combinations thereof.

11. A method of activating an immune cell in a subject, the method comprising administering to the subject a CYR61 activating agent.

12. The method of claim 11, wherein the subject has ocular neovascularization, macular degeneration, age-related macular degeneration (AMD), a retinal disease, or diabetic retinopathy.

13. The method of claim 12, wherein the CYR61 activating agent is CYR61.

14. The method of claim 11, wherein the immune cell is selected from a macrophage, a monocyte, a monocyte-derived macrophage, a bone marrow-derived macrophage (BMDM), and a choroidal macrophage.

15. The method of claim 11, wherein the subject has at least one choroidal neovascular (CNV) lesion.

16. The method of claim 15, wherein administering the CYR61 activating agent to the subject reduces CNV lesion size.

17. The method of claim 11, further comprising administering to the subject at least one of an anti-VEGF treatment and a photodynamic therapy.

18. The method of claim 11, wherein the CYR61 activating agent is administered to the subject intravitreally.

19. The method of claim 11, wherein administering the CYR61 activating agent to the subject upregulates immune cell expression of one or more genes selected from the group consisting of Saa3, Mmp9, Gm14493, Cfb, Mmp14, Slpi, Gm49383, Marco, Rps12I1, Pilra, Hp, CcI5, Nxpe5, Acod1, C3, Socs3, Mid1-ps1, Il1b, and combinations thereof.

20. The method of claim 1, wherein administering the CYR61 activating agent to the subject results in at least one of:
upregulating endothelial cell expression of one or more genes selected from the group consisting of CCL2, CXCL6, CXCL1, TFRC, IL32, and combinations thereof; and
downregulating endothelial cell expression of one or more genes selected from the group consisting of LYVE1, DEPP1, FCF1, AC020916.1, LINC02603, and combinations thereof.
